(19) United States
(12) Patent Application Publication
(10) Pub. No.: US 2024/0025845 A1
(43) Pub. Date: Jan. 25, 2024

(54) METASTASIS-INHIBITING COMPOSITION OF NOVEL METHYLSULFONAMIDE DERIVATIVE COMPOUND

(71) Applicant: KOREA RESEARCH INSTITUTE OF BIOSCIENCE AND BIOTECHNOLOGY, Daejeon (KR)

(72) Inventors: Jong Seog AHN, Daejeon (KR); Sung Kyun KO, Daejeon (KR); Jae Hyuk JANG, Daejeon (KR); Jun Pil JANG, Daejeon (KR); Jae Kyung JUNG, Cheongju-si, Chungcheongbuk-do (KR); Nam Doo KIM, Incheon (KR); Bo Yeon KIM, Daejeon (KR); Joon Suk PARK, Daegu (KR)

(21) Appl. No.: 18/025,134

(22) PCT Filed: Sep. 7, 2021
(86) PCT No.: PCT/KR2021/012074

§ 371 Date: Mar. 7, 2023

(30) Foreign Application Priority Data
Sep. 7, 2020 (KR) 10-2020-0113870

(51) Int. Cl. C07C 311/08; C07C 237/52; C07D 209/48; C07D 333/34; C07D 213/64; A61P 35/00; C07C 311/44; C07C 311/29; C07C 311/21; C07D 333/18; C07D 231/16; C07D 215/36; C07D 403/04; C07C 311/14; C07D 271/12; C07D 319/18; C07C 311/13

(52) U.S. Cl. CPC C07C 311/08; C07C 237/52; C07D 209/48; C07D 333/34; C07D 213/64; A61P 35/00; C07C 311/44; C07C 311/29; C07C 311/21; C07D 333/18; C07D 231/16; C07D 215/36; C07D 403/04; C07C 311/14; C07D 271/12; C07D 319/18; C07C 311/13; C07C 2601/02

(57) ABSTRACT

Provided is a metastasis-inhibiting composition containing a methylsulfonamide-based derivative compound as an active ingredient. More specifically, the compounds inhibit the activity of chromosome segregation 1-like (CSE1L), and suppress intracellular nuclear transport to inhibit the movement and/or invasion of cancer cells, and thus effectively inhibit metastasis.

### TECHNICAL FIELD

The present invention relates to a use of a methylsulfonamide-based derivative compound to inhibit metastasis, and more specifically, to a use of inhibiting metastasis by inhibiting chromosome segregation 1-like (CSE1L) activity and inhibiting intracellular nuclear transport.

### BACKGROUND ART

Recently, interest in health has increased due to an increase in life expectancy and the desire for a healthy life has increased as the quality of life has improved. The life has extended due to improvement in quality of life and improved health care, but on the other hand, chronic diseases such as cancer, diabetes, mental illness, and heart disease have increased. Until now, various methods and the like have been attempted to treat these chronic diseases, and in particular, many methods for treating cancer have been developed. Among them, as representative methods of treating cancer, methods such as drugs, surgery, and radiation have been used. Among these methods, there is a method of killing cancer cells by directly injecting orally or intravenously to kill cancer cells by treating a drug that selectively acts only on cancer cells, but in this case, many side effects are caused by administering a large amount of the drug. Accordingly, efforts are being made to develop safe substances.

Meanwhile, metastasis refers to a state in which cancer in a new tissue occurs by leaving cancer cells from an organ or tissue which first occurred to infiltrate and proliferate the cancer cells in another organ or tissue. Methods of metastasis include a contact metastasis, a hematogenous metastasis via blood vessels, and a lymphogenous metastasis via lymphatic vessels. When the cancer leaves a primary origin site and progresses to metastasis, new cancer tissue is formed in various organs, and treatment by anticancer drugs, surgery, or radiation becomes difficult, leading to death. Accordingly, preventing metastasis of the primary cancer is very important in cancer treatment, but until now, development of metastasis inhibitors that prevent metastasis of cancer has not yet been achieved.

The nucleus is surrounded by a nuclear membrane to be separated from the cytoplasm, and the nuclear membrane has nuclear pores to serve as passages for necessary substances or proteins, enabling movement of substances between the intra-nucleus and the cytoplasm. However, generally, molecules with a molecular weight of less than 40 kD may pass through the nuclear pores, but macromolecules with a molecular weight of 40 kD or more may not pass through the nuclear pores on their own, and thus bind to a nuclear transport protein passing through the nuclear pores to move from the cytoplasm to the nucleus or from the intra-nucleus to the cytoplasm with the help thereof. Importin, a nuclear import protein, which passes through the nuclear pores from the cytoplasm into the nucleus, has importin α, importin β, and the like (Chook, Y. M.; Blobel, G. Curr. Opin. Struct. Biol. 2001, 11, 703). Exportin, a nuclear export protein, which passes through the nuclear pores from the intra-nucleus to the cytoplasm, is known as exportin 1 (Crm1), exportin 2 (CSE1L; chromosome segregation 1-like), exportin t, exportin 4, exportin 5, exportin 6, exportin 7, and the like (Pemberton, L. F.; Paschal, B. M. Traffic 2005, 6, 187).

In general, in the protein transport from the cytoplasm into the nucleus, importin α recognizes and binds to a cargo protein to move into the nucleus, which has a position-recognizing amino acid sequence in the nucleus, and additionally binds to importin β to form a tertiary complex, and the complex passes through the nuclear pores and then in the nucleus, a Ran protein (Ran-GTP) binds to importin β, and the importin α and cargo proteins are separated so that the movement into the nucleus is completed.

The cargo protein that has moved into the nucleus functions in the nucleus and moves back to the cytoplasm with the help of exportin 1 if necessary, and the importin β in the nucleus has a structure that may pass through its own nuclear core to move into the cytoplasm. However, in the case of importin α, when moving from the intra-nucleus to the cytoplasm, importin ca cannot pass through the nuclear pores by itself, and thus binds to exportin 2, CSE1L to move to the cytoplasm with the help thereof and is reused for protein movement from the cytoplasm back into the nucleus (Solsbacher, J.; Maurer, P.; Bischoff, F. R.; Schlenstedt, G. Mol. Cell. Biol. 1998, 18, 6805). Specifically, in the case of movement of importin a from the intra-nucleus to the cytoplasm, the GTP-bound Ran protein (Ran-GTP) binds to CSE1L by a high concentration of GTP present in the nucleus to attach importin α and moves to the cytoplasm through the nuclear pores. The Ran-GTP protein bound to CSE1L released into the cytoplasm is rapidly hydrolyzed to Ran GDP and then importin α is isolated from the cytoplasm while the structure to a cargo free state (a state in which importin-α is not bound), and if necessary, importin α, which moves the cytoplasmic protein into the nucleus, serves to be circulated.

It has been known that the CSE1L protein, which has the functions, is highly present in cancer cells or tissues to play a role in a carcinogenesis process, and particularly, as can be seen in reports that the CSE1L protein is involved in functions such as movement and invasion of cancer cells and a particularly high expression rate is shown in cancer cell lines with high metastatic ability or in metastatic cancer cells of patients with metastasis (Stella Tsai, C.-S.; Chen, H.-C.; Tung, J.-N.; Tsou, S.-S.; Tsao, T.-Y.; Liao, C.-F.; Chen, Y.-C.; Yeh, C.-Y.; Yeh, K.-T.; Jiang, M.-C. The American Journal of Pathology 2010, 176, 1619.), the role and function of the CSE1L protein in metastasis have been reported. However, an exact mechanism has not yet been reported.

Accordingly, the present inventors developed a compound that binds to the CSE1L protein to selectively modulate the function of CSE1L, confirmed that metastasis was inhibited by the compound, and then completed the present invention.

### DISCLOSURE

### Technical Problem

An object of the present invention is to provide a methylsulfonamide-based compound, a stereoisomer thereof, a tautomer thereof, or a pharmaceutically acceptable salt thereof.

Another object of the present invention is to provide a composition for preventing or treating cancer containing a methylsulfonamide-based compound or a pharmaceutically acceptable salt thereof as an active ingredient.

Yet another object of the present invention is to provide a metastasis-inhibiting composition containing a methylsulfonamide-based compound or a pharmaceutically acceptable salt thereof as an active ingredient.

Still another object of the present invention is to provide an angiogenesis-inhibiting composition containing a methylsulfonamide-based compound or a pharmaceutically acceptable salt thereof as an active ingredient.

### Technical Solution

One aspect of the present invention provides a methylsulfonamide derivative compound represented by Chemical Formula 1 below, a stereoisomer thereof, or a pharmaceutically acceptable salt thereof.

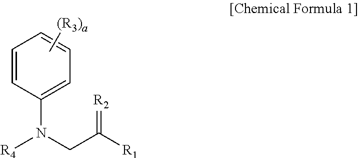

Another aspect of the present invention provides a composition for preventing or treating cancer comprising a compound represented by Chemical Formula 1, a stereoisomer thereof, or a pharmaceutically acceptable salt thereof as an active ingredient.

Another aspect of the present invention provides a metastasis-inhibiting composition comprising a compound represented by Chemical Formula 1, a stereoisomer thereof, or a pharmaceutically acceptable salt thereof as an active ingredient.

Another aspect of the present invention provides an angiogenesis-inhibiting composition comprising a compound represented by Chemical Formula 1, a stereoisomer thereof, or a pharmaceutically acceptable salt thereof as an active ingredient.

Another aspect of the present invention provides a kit for preventing or treating cancer comprising a first ingredient containing the compound represented by Chemical Formula 1, a stereoisomer thereof, or a pharmaceutically acceptable salt thereof as an active ingredient; and a second ingredient containing an anticancer agent as an active ingredient.

Another aspect of the present invention provides a metastasis-inhibiting kit comprising a first ingredient containing the compound represented by Chemical Formula 1, a stereoisomer thereof, or a pharmaceutically acceptable salt thereof as an active ingredient; and a second ingredient containing a metastasis-inhibiting agent as an active ingredient.

Another aspect of the present invention provides an angiogenesis-inhibiting kit comprising a first ingredient containing the compound represented by Chemical Formula 1, a stereoisomer thereof, or a pharmaceutically acceptable salt thereof as an active ingredient; and a second ingredient containing an angiogenesis-inhibiting ingredient as an active ingredient.

Another aspect of the present invention provides a method for preventing or treating cancer comprising administering the compound represented by Chemical Formula 1, a stereoisomer thereof, or a pharmaceutically acceptable salt thereof to a subject in need thereof in a therapeutically effective amount.

Another aspect of the present invention provides a metastasis-inhibiting method comprising administering the compound represented by Chemical Formula 1, a stereoisomer thereof, or a pharmaceutically acceptable salt thereof to a subject in need thereof in a therapeutically effective amount.

Another aspect of the present invention provides an angiogenesis-inhibiting method comprising administering the compound represented by Chemical Formula 1, a stereoisomer thereof, or a pharmaceutically acceptable salt thereof to a subject in need thereof in a therapeutically effective amount.

Another aspect of the present invention provides the compound represented by Chemical Formula 1, a stereoisomer thereof, or a pharmaceutically acceptable salt thereof for use in prevention or treatment of cancer.

Another aspect of the present invention provides the compound represented by Chemical Formula 1, a stereoisomer thereof, or a pharmaceutically acceptable salt thereof for use in metastasis inhibiting.

Another aspect of the present invention provides the compound represented by Chemical Formula 1, a stereoisomer thereof, or a pharmaceutically acceptable salt thereof for use in angiogenesis inhibition.

Another aspect of the present invention provides a use of the compound represented by Chemical Formula 1, a stereoisomer thereof, or a pharmaceutically acceptable salt thereof for preparing a medicament for use in prevention or treatment of cancer.

Another aspect of the present invention provides a use of the compound represented by Chemical Formula 1, a stereoisomer thereof, or a pharmaceutically acceptable salt thereof for preparing a medicament for use in metastasis inhibition.

Another aspect of the present invention provides a use of the compound represented by Chemical Formula 1, a stereoisomer thereof, or a pharmaceutically acceptable salt thereof for preparing a medicament for use in angiogenesis inhibition.

Another aspect of the present invention provides a composition comprising the compound represented by Chemical Formula 1 or a pharmaceutically acceptable salt thereof and a pharmaceutically acceptable excipient.

### Advantageous Effects

According to the present invention, the compound can inhibit intracellular nuclear transport by effectively inhibiting a function of CSE1L without cytotoxicity and effectively inhibit the movement and invasion of cancer cells, and accordingly, the composition containing the compound according to the present invention as an active ingredient can be used as an anti-cancer therapeutic agent that effectively inhibits metastasis.

### DESCRIPTION OF DRAWINGS

FIGS. 1A-1B illustrate the discovery of a methylsulfonamide-based compound, which is a novel compound of a metastasis-targeting protein CSE1L, using a molecular modeling technique of the present invention.

FIGS. 2A-2E illustrate the cytotoxicity, cancer cell movement and motility regulation activity analysis of a compound 1-1 of the present invention.

FIGS. 3A-3C illustrate results of evaluating in vivo toxicity using a metastasis breast cancer model (Orthotopic xenograft spontaneous metastasis mouse model) of the compound 1-1 of the present invention, verifying a metastasis-inhibiting effect through bioluminescence, and confirming the number of lung metastasis through autopsy.

FIGS. 4A-4B illustrate the active analysis of cytotoxicity of compounds 1-1 to 1-47 of the present invention.

FIGS. 5A-5B illustrate verification analysis of cell movement inhibitory activity and in vivo stability of the compounds 1-1 to 1-47 of the present invention.

FIGS. 6A-6C illustrate the cytotoxicity, cancer cell movement and motility regulation activity assay of a compound 1-46 of the present invention.

FIGS. 7A-7C illustrate results of using a chorioallantoic membrane (CAM) technique for verifying the angiogenesis inhibitory activity of the compound 1-46 of the present invention.

### BEST MODE OF THE INVENTION

Hereinafter, examples of the present invention will be described in detail. In the following description, a detailed explanation of related known configurations or functions may be omitted to avoid obscuring the subject matter of the present invention.

The present invention provides a compound represented by Chemical Formula 1 below, a stereoisomer thereof, a tautomer thereof, or a pharmaceutically acceptable salt thereof.

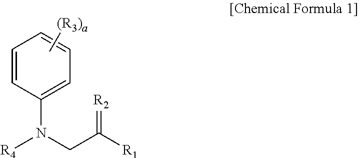

In Chemical Formula 1, each symbol may be defined as follows.
1) R₁ is OH, NH—R₅ or O—R₆.
2) The R₅ is H, OH, NH₂ or a C₁-C₁₀ alkyl group.

When the R₅ is an alkyl group, the R₅ may be preferably a C₁-C₆ alkyl group, more preferably a C₁-C₃ alkyl group.
3) The R₆ may be a C₁-C₁₀ alkyl group, preferably a C₁-C₆ alkyl group, more preferably a C₁-C₄ alkyl group.
4) R₂ is O or absent.
5) R₃ is the same as or different from each other, and is selected from the group consisting of hydrogen; halogen; a C₁-C₁₀ alkyl group; a C₁-C₁₀ alkoxy group; a C₁-C₁₀ alkyl group substituted with fluorine; and a C₁-C₁₀ alkoxy group substituted with fluorine.

When the R₃ is an alkyl group, the R₃ may be preferably a C₁-C₆ alkyl group, more preferably a C₁-C₃ alkyl group.

When the R₃ is an alkoxy group, the R₃ may be preferably a C₁-C₆ alkoxy group, more preferably a C₁-C₃ alkoxy group.

When the R₃ is the alkyl group substituted with fluorine, the R₃ may be preferably a C₁-C₆ alkyl group substituted with fluorine, more preferably a C₁-C₃ alkyl group substituted with fluorine.

When the R₃ is the alkoxy group substituted with fluorine, the R₃ may be preferably a C₁-C₆alkoxy group substituted with fluorine, more preferably a C₁-C₃ alkoxy group substituted with fluorine.
6) a is an integer of 0 to 5.
7) R₄ is —SO₂—R₇; or —CO₂—(CH₂)ₘ—R₈.
8) The R₇ is selected from the group consisting of a C₁-C₁₀ alkyl group; a C₁-C₁₀ alkyl group substituted with fluorine; a C₃-C₁₀ cycloalkyl group; a C₆-C₂₄ aryl group; a C₂-C₂₄ heterocyclic group; and —(CH₂)ₙ—R₉.

When the R₇ is the alkyl group, the R₇ may be preferably a C₁-C₆ alkyl group, more preferably a C₁-C₃ alkyl group.

When the R₇ is the alkyl group substituted with fluorine, the R₇ may be preferably a C₁-C₆ alkyl group substituted with fluorine, more preferably a C₁-C₃ alkyl group substituted with fluorine.

When the R₇ is the cycloalkyl group, the R₇ may be preferably a C₃-C₆ cycloalkyl group, more preferably a C₁-C₃ cycloalkyl group.

When the R₇ is the aryl group, the R₇ may be preferably a C₆-C₁₈ aryl group, more preferably a C₆-C₁₂ aryl group.

When the R₇ is the heterocyclic group, the R₇ may be preferably a C₆-C₁₅ heterocyclic group, more preferably a C₂-C₁₀ heterocyclic group.
9) The R₈ and R₉ are each independently a C₆-C₂₄ aryl group; or a C₂-C₂₄ heterocyclic group.

When the R₈ and R₉ are the aryl groups, the R₈ and R₉ may be preferably C₆-C₁₈ aryl groups, more preferably C₆-C₁₂ aryl groups.

When the R₈ and R₉ are the heterocyclic groups, the R₈ and R₉ may be preferably C₂-C₁₅ heterocyclic groups, more preferably C₂-C₁₀ heterocyclic groups.
10) m and n are independently integers of 0 to 5.
11) Here, each of the alkyl group, alkoxy group, cycloalkyl group, aryl group, and heterocyclic group may be further substituted with at least one substituent selected from the group consisting of halogen; a C₁-C₁₀ alkyl group; a C₁-C₁₀ alkyl group substituted with halogen; a C₆-C₁₂ aryl group substituted with halogen; a C₂-C₁₀ heterocyclic group; a C₂-C₁₀ heterocyclic group substituted with halogen; a C₂-C₁₀ heterocyclic group substituted with CF₃; —NR^{a}R^{b}; a —SO₂-phenyl group; a C₆-C₁₂ aryloxy group; a C₂-C₁₂ heteroaryloxy group; and a C₂-C₁₂ heteroaryloxy group substituted with CF₃, and
12) the R^{a} and R^{b} are each independently C₁-C₁₀ alkyl groups.}

In addition, in the present invention, the compound represented by Chemical Formula 1 includes a compound represented by Chemical Formula 2 below.

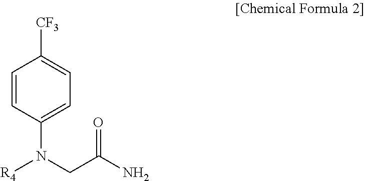

{In Chemical Formula 2, R₄ is the same as the definition of R₄ in Chemical Formula 1.}

In addition, in the present invention, the compound represented by Chemical Formula 1 includes any one represented by compounds 1-1 to 1-47 below.

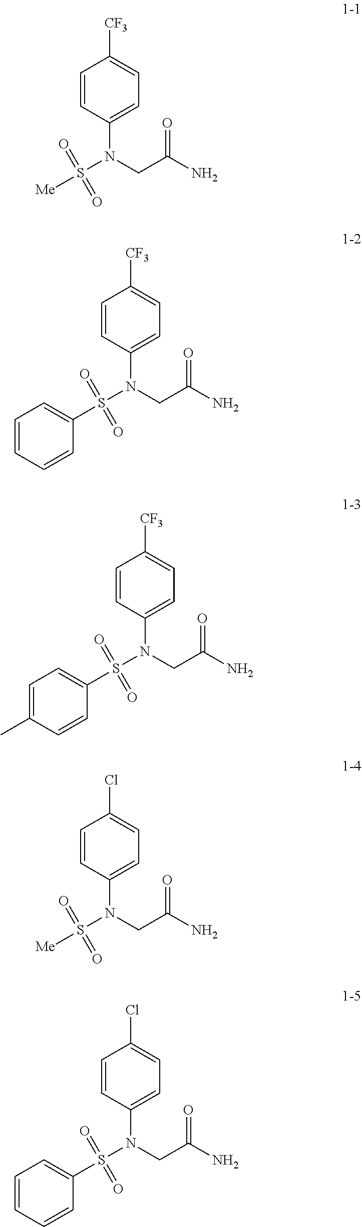 [SMILES: CS(=O)(=O)N(CC(N)=O)c1ccc(C(F)(F)F)cc1 ; NC(=O)CN(c1ccc(C(F)(F)F)cc1)S(=O)(=O)c1ccccc1 ; Cc1ccc(S(=O)(=O)N(CC(N)=O)c2ccc(C(F)(F)F)cc2)cc1 ; CS(=O)(=O)N(CC(N)=O)c1ccc(Cl)cc1 ; NC(=O)CN(c1ccc(Cl)cc1)S(=O)(=O)c1ccccc1]

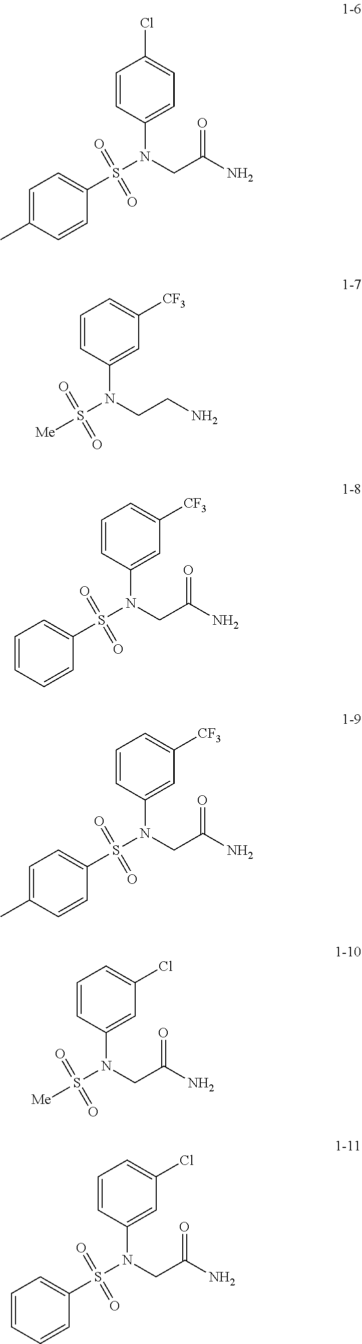 [SMILES: Cc1ccc(S(=O)(=O)N(CC(N)=O)c2ccc(Cl)cc2)cc1 ; CS(=O)(=O)N(CCN)c1cccc(C(F)(F)F)c1 ; NC(=O)CN(c1cccc(C(F)(F)F)c1)S(=O)(=O)c1ccccc1 ; Cc1ccc(S(=O)(=O)N(CC(N)=O)c2cccc(C(F)(F)F)c2)cc1 ; CS(=O)(=O)N(CC(N)=O)c1cccc(Cl)c1 ; NC(=O)CN(c1cccc(Cl)c1)S(=O)(=O)c1ccccc1]

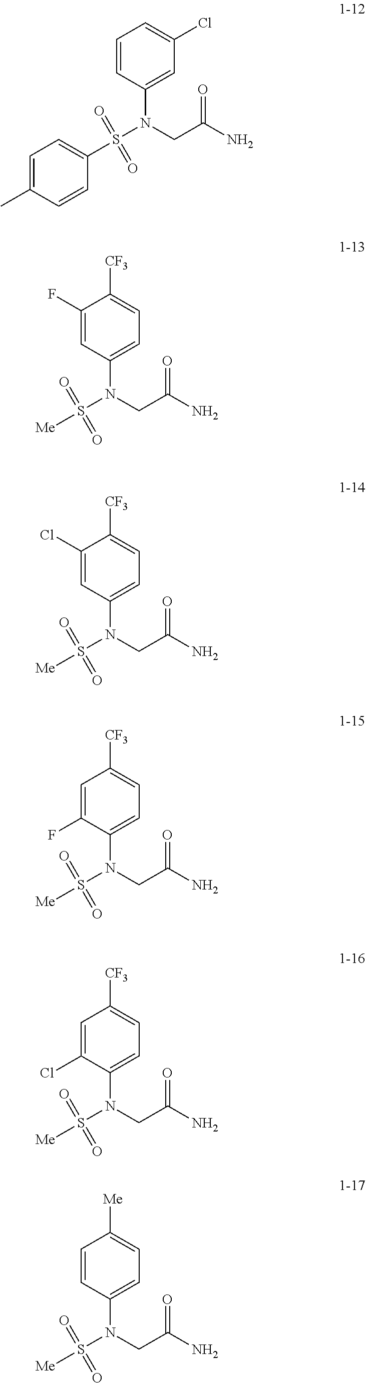 [SMILES: Cc1ccc(S(=O)(=O)N(CC(N)=O)c2cccc(Cl)c2)cc1 ; CS(=O)(=O)N(CC(N)=O)c1ccc(C(F)(F)F)c(F)c1 ; CS(=O)(=O)N(CC(N)=O)c1ccc(C(F)(F)F)c(Cl)c1 ; CS(=O)(=O)N(CC(N)=O)c1ccc(C(F)(F)F)cc1F ; CS(=O)(=O)N(CC(N)=O)c1ccc(C(F)(F)F)cc1Cl ; Cc1ccc(N(CC(N)=O)S(C)(=O)=O)cc1]

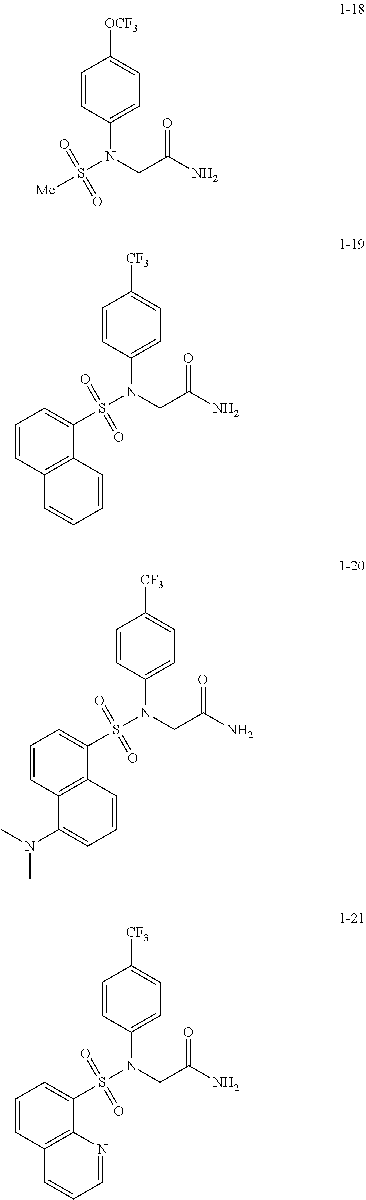 [SMILES: CS(=O)(=O)N(CC(N)=O)c1ccc(OC(F)(F)F)cc1 ; NC(=O)CN(c1ccc(C(F)(F)F)cc1)S(=O)(=O)c1cccc2ccccc12 ; CN(C)c1cccc2c(S(=O)(=O)N(CC(N)=O)c3ccc(C(F)(F)F)cc3)cccc12 ; NC(=O)CN(c1ccc(C(F)(F)F)cc1)S(=O)(=O)c1cccc2cccnc12]

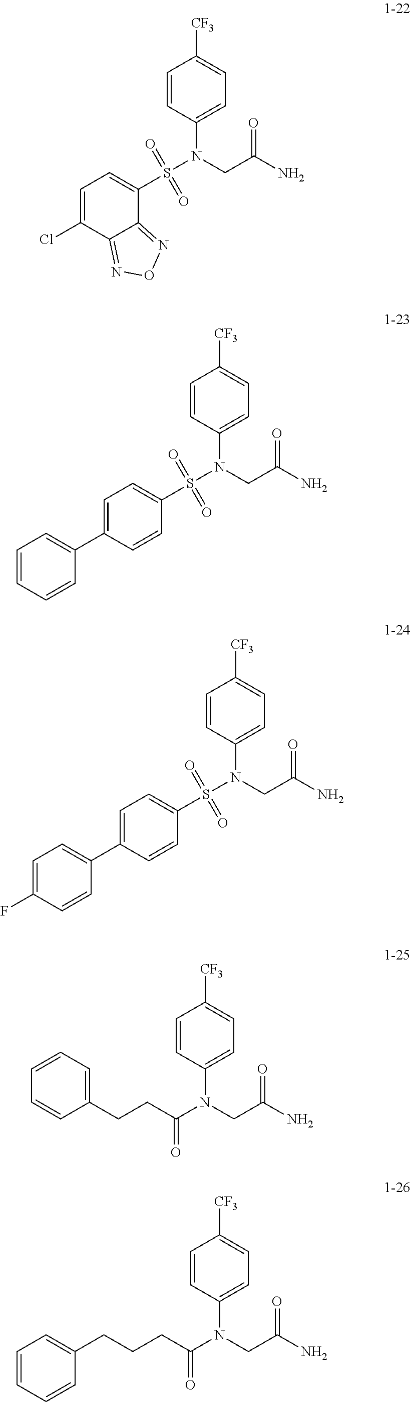 [SMILES: NC(=O)CN(c1ccc(C(F)(F)F)cc1)S(=O)(=O)c1ccc(Cl)c2nonc12 ; NC(=O)CN(c1ccc(C(F)(F)F)cc1)S(=O)(=O)c1ccc(-c2ccccc2)cc1 ; NC(=O)CN(c1ccc(C(F)(F)F)cc1)S(=O)(=O)c1ccc(-c2ccc(F)cc2)cc1 ; NC(=O)CN(C(=O)CCc1ccccc1)c1ccc(C(F)(F)F)cc1 ; NC(=O)CN(C(=O)CCCc1ccccc1)c1ccc(C(F)(F)F)cc1]

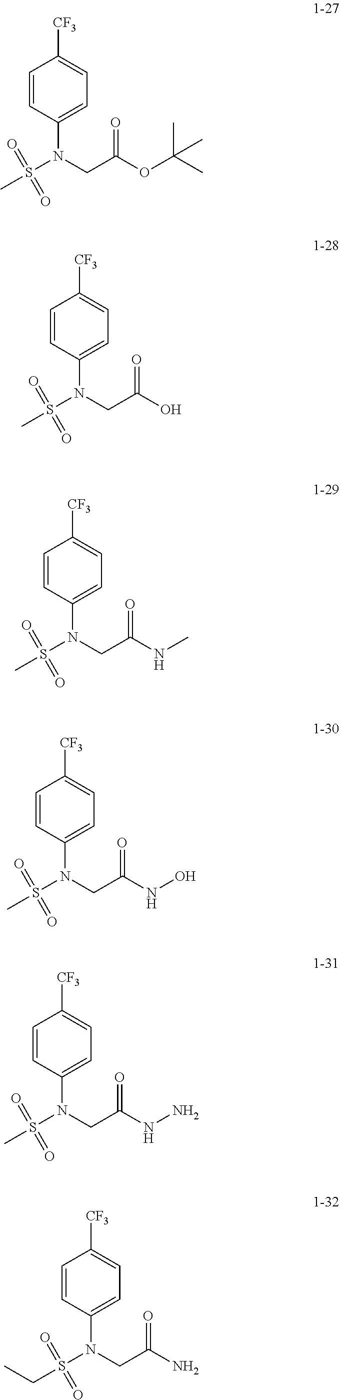

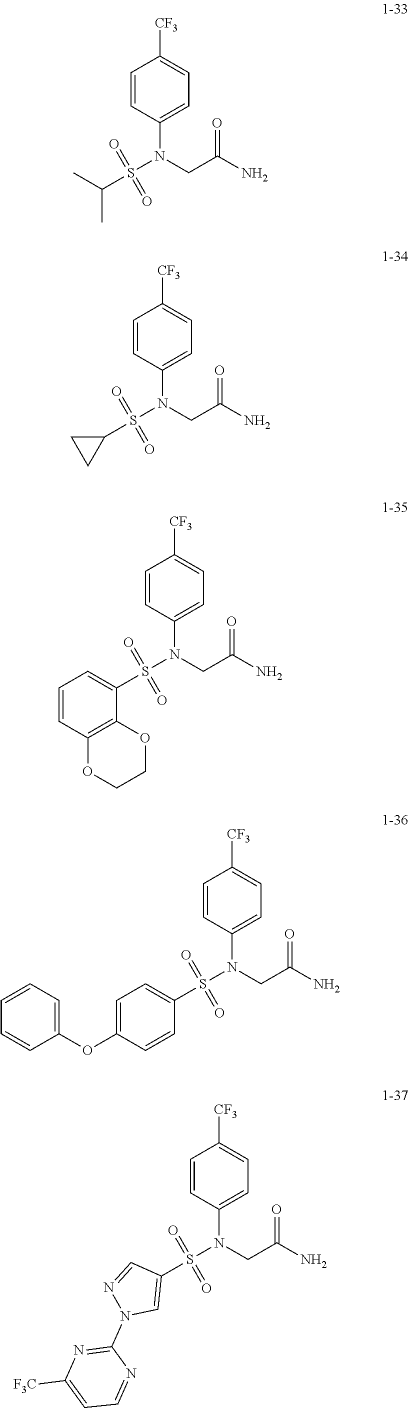 [SMILES: CC(C)S(=O)(=O)N(CC(N)=O)c1ccc(C(F)(F)F)cc1 ; NC(=O)CN(c1ccc(C(F)(F)F)cc1)S(=O)(=O)C1CC1 ; NC(=O)CN(c1ccc(C(F)(F)F)cc1)S(=O)(=O)c1cccc2c1OCCO2 ; NC(=O)CN(c1ccc(C(F)(F)F)cc1)S(=O)(=O)c1ccc(Oc2ccccc2)cc1 ; NC(=O)CN(c1ccc(C(F)(F)F)cc1)S(=O)(=O)c1cnn(-c2nccc(C(F)(F)F)n2)c1]

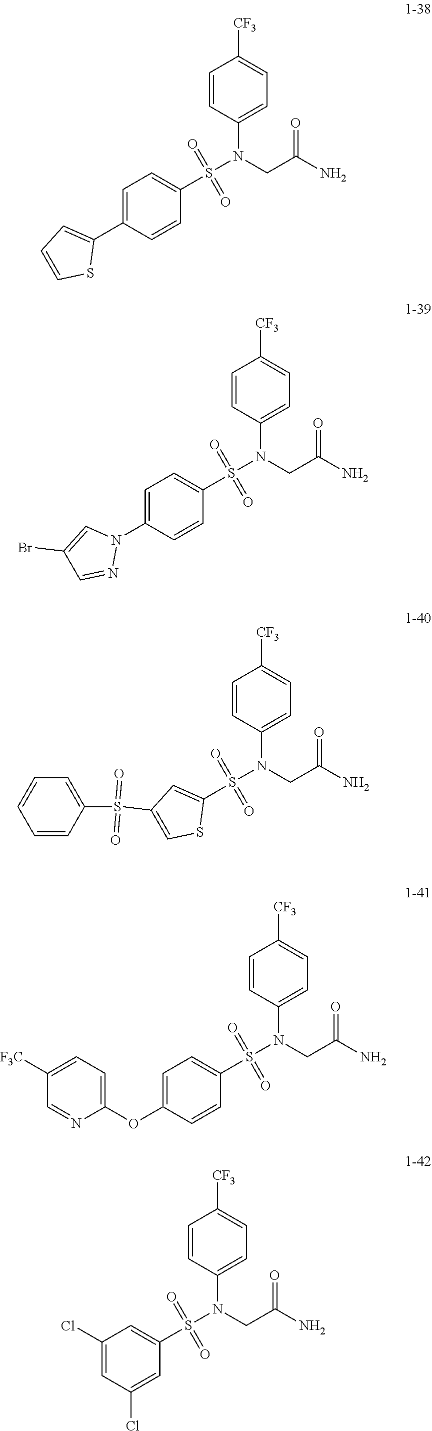 [SMILES: NC(=O)CN(c1ccc(C(F)(F)F)cc1)S(=O)(=O)c1ccc(-c2cccs2)cc1 ; NC(=O)CN(c1ccc(C(F)(F)F)cc1)S(=O)(=O)c1ccc(-n2cc(Br)cn2)cc1 ; NC(=O)CN(c1ccc(C(F)(F)F)cc1)S(=O)(=O)c1cc(S(=O)(=O)c2ccccc2)cs1 ; NC(=O)CN(c1ccc(C(F)(F)F)cc1)S(=O)(=O)c1ccc(Oc2ccc(C(F)(F)F)cn2)cc1 ; NC(=O)CN(c1ccc(C(F)(F)F)cc1)S(=O)(=O)c1cc(Cl)cc(Cl)c1]

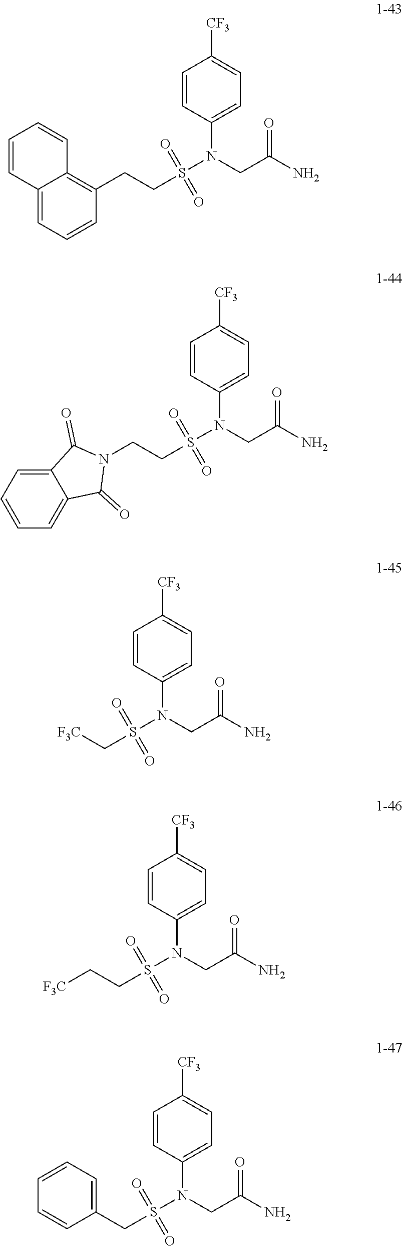 [SMILES: NC(=O)CN(c1ccc(C(F)(F)F)cc1)S(=O)(=O)CCc1cccc2ccccc12 ; NC(=O)CN(c1ccc(C(F)(F)F)cc1)S(=O)(=O)CCN1C(=O)c2ccccc2C1=O ; NC(=O)CN(c1ccc(C(F)(F)F)cc1)S(=O)(=O)CC(F)(F)F ; NC(=O)CN(c1ccc(C(F)(F)F)cc1)S(=O)(=O)CCC(F)(F)F ; NC(=O)CN(c1ccc(C(F)(F)F)cc1)S(=O)(=O)Cc1ccccc1]

In addition, in another aspect, the present invention provides a composition for preventing or treating cancer comprising the compound represented by Chemical Formula 1 or a pharmaceutically acceptable salt thereof as an active ingredient.

In addition, in another aspect, the present invention provides a metastasis-inhibiting composition comprising the compound represented by Chemical Formula 1 or a pharmaceutically acceptable salt thereof as an active ingredient.

In the composition for preventing or treating cancer and the metastasis-inhibiting composition, the cancer may be selected from the group consisting of liver cancer, colorectal cancer, cervical cancer, kidney cancer, gastric cancer, prostate cancer, breast cancer, brain tumor, lung cancer, colon cancer, bladder cancer, and pancreatic cancer.

In addition, in another aspect, the present invention provides an angiogenesis-inhibiting composition comprising the compound represented by Chemical Formula 1 or a pharmaceutically acceptable salt thereof as an active ingredient.

The angiogenesis may be at least one selected from the group consisting of rheumatoid arthritis, osteoarthritis, septic arthritis, psoriasis, corneal ulcers, aging-related macular degeneration, diabetic retinopathy, proliferative vitreoretinopathy, immature retinopathy, ophthalmic inflammation, keratoconus, Sjogren's syndrome, myopic eye tumor, corneal transplant rejection, abnormal wound closure, bone disease, proteinuria, abdominal aortic aneurysm disease, degenerative cartilage loss due to traumatic joint injury, demyelinating disease of the nervous system, liver cirrhosis, glomerular disease, immature rupture of embryonic membrane, inflammatory bowel disease, periodontal disease, arteriosclerosis, restenosis, inflammatory disease of the central nervous system, Alzheimer's disease, skin aging, and cancer invasion and metastasis.

The compounds of the present invention may exist in the form of pharmaceutically acceptable salts. As the salts, acid addition salts formed with pharmaceutically acceptable free acids are useful. The term “pharmaceutically acceptable salt” used herein refers to any organic or inorganic addition salt of the compound in which side effects caused by the salt does not degrade the beneficial effect of the compound according to the present invention as a concentration that is relatively non-toxic and has a harmless effective effect on a patient.

The acid addition salt is prepared by a general method, for example, by dissolving a compound in an excess acid aqueous solution and precipitating the salt using a water-miscible organic solvent, such as methanol, ethanol, acetone or acetonitrile. The same molar amount of compound and acid or alcohol (e.g., glycol monomethyl ether) in water are heated, and then the mixture may be evaporated and dried, or the precipitated salt may be suction-filtered.

At this time, as the free acid, organic acids and inorganic acids may be used. As the inorganic acids, hydrochloric acid, phosphoric acid, sulfuric acid, nitric acid, bromic acid, iodic acid, perchloric acid, or the like may be used, and as the organic acids, methanesulfonic acid, p-toluenesulfonic acid, acetic acid, trifluoroacetic acid, maleic acid, succinic acid, oxalic acid, benzoic acid, tartaric acid, fumaric acid, mandelic acid, propionic acid, citric acid, lactic acid, glycolic acid, gluconic acid, galacturonic acid, glutamic acid, glutaric acid, glucuronic acid, aspartic acid, ascorbic acid, carbonic acid, vanillic acid, or the like may be used. However, the acids are not limited thereto.

Further, pharmaceutically acceptable metal salts may be prepared using bases. An alkali metal salt or an alkaline earth metal salt may be obtained, for example, by dissolving the compound in a large amount of alkali metal hydroxide or alkaline earth metal hydroxide solution, filtering a non-dissolved compound salt, and then evaporating and drying a filtrate. In this case, as the metal salt, salts prepared by, particularly, sodium, potassium or calcium are pharmaceutically suitable, but are not limited thereto. Further, the silver salt corresponding thereto may be obtained by reacting alkali metal or alkaline earth metal salts with an appropriate silver salt (e.g., silver nitrate).

The salt of the methylsulfonamide-based compound of the present invention is a pharmaceutically acceptable salt, and any salt of methylsulfonamide-based compounds of compounds 1-1 to 1-47 may be used without limitation.

Since the compounds 1-1 to 1-47 of the present invention or pharmaceutically acceptable salts thereof may inhibit the movement and invasion functions of cancer cells, the compounds may be usefully used for anticancer treatment through inhibition of metastasis.

The methylsulfonamide-based compounds represented by the compounds 1-1 to 1-47 of the present invention, or pharmaceutically acceptable salts thereof suppress and regulate the activity of chromosome segregation 1-like (CSE1L) to inhibit intracellular nuclear transport, and particularly may inhibit the movement and invasion functions of cancer cells, and thus may be usefully used for the prevention and treatment of cancer through inhibition of metastasis.

Specifically, the composition of the present invention may usefully inhibit metastasis of solid cancers selected from the group consisting of, for example, liver cancer, colorectal cancer, cervical cancer, kidney cancer, gastric cancer, prostate cancer, breast cancer, brain tumor, lung cancer, uterine cancer, colon cancer, bladder cancer and pancreatic cancer. However, the metastasis inhibited by the pharmaceutical composition of the present invention is not limited to the cancers.

In the present invention, the term “prevention” refers to all activities that inhibit or delay the occurrence, spread, and recurrence of CSE1L-related metastasis by administration of the composition of the present invention, and the term “treatment” refers to all activities that improve or beneficially change the symptoms of the metastasis by administration of the composition of the present invention.

In addition, the composition of the present invention may further include a pharmaceutically acceptable carrier, a diluent, or an excipient. The composition of the present invention may be formulated and used in various forms such as oral formulations such as powders, granules, tablets, capsules, suspensions, emulsions, syrups, aerosols, and injections of sterile injection solutions according to conventional methods according to each purpose of use, and may be administered orally or administered through various routes including intravenous, intraperitoneal, subcutaneous, rectal, topical, and the like. Examples of suitable carriers, excipients, or diluents, which may be included in the composition, may include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, mineral oil, and the like. In addition, the composition of the present invention may further include fillers, anti-coagulating agents, lubricants, wetting agents, flavorings, emulsifiers, preservatives, and the like.

Solid formulations for oral administration include a tablet, a pill, a powder, a granule, a capsule, and the like, and the solid formulations may be prepared by mixing at least one excipient, for example, starch, calcium carbonate, sucrose, lactose, gelatin, and the like with the composition. Further, lubricants such as magnesium stearate and talc may be used in addition to simple excipients.

Liquid formulations for oral administration may include suspensions, oral liquids, emulsions, syrups, and the like, and may include various excipients, for example, a wetting agent, a sweetener, an aromatic agent, a preservative, and the like, in addition to water and liquid paraffin which are commonly used as simple diluents.

Formulations for parenteral administration include sterilized aqueous solutions, non-aqueous solvents, suspensions, emulsions, lyophilized agents, and suppositories. As the non-aqueous solution and the suspension, propylene glycol, polyethylene glycol, vegetable oil such as olive oil, injectable ester such as ethyl oleate, and the like may be used. As a base of the suppository, witepsol, macrogol, Tween 61, cacao butter, laurinum, glycerogelatin, and the like may be used. Meanwhile, the injections may include conventional additives, such as solubilizers, isotonic agents, suspending agents, emulsifiers, stabilizers and preservatives.

The composition of the present invention is administered in a pharmaceutically effective dose. In the present invention, the term “pharmaceutically effective dose” refers to an amount enough to treat the disease at a reasonable benefit/risk ratio applicable to medical treatment and does not cause side effects. An effective dose level may be determined according to factors including the health condition of a patient, the type and severity of a disease, the activity of a drug, the sensitivity to a drug, a method of administration, a time of administration, a route of administration, an excretion rate, duration of treatment, and drugs used in combination and simultaneously, and other factors well-known in the medical field. The composition of the present invention may be administered as an individual therapeutic agent or in combination with other therapeutic agents, and may be administered sequentially or simultaneously with existing therapeutic agents, and may be administered singly or multiply. It is important to administer an amount capable of obtaining a maximum effect with a minimal amount without side effects by considering all the factors, which may be easily determined by those skilled in the art.

According to another aspect of the present invention, the present invention provides a method for preventing or treating metastasis comprising administering a compound represented by Chemical Formula 1, a stereoisomer thereof, a tautomer thereof, or a pharmaceutically acceptable salt thereof to a subject in need thereof, such as a human or non-human mammal.

In the present invention, the term “subject” refers to all animals, including monkeys, cows, horses, sheep, pigs, chickens, turkeys, quails, cats, dogs, mice, rats, rabbits or guinea pigs including humans, which have developed or may develop metastasis. It is possible to effectively prevent or treat the disease by administering the compound of the present invention to the subject. The compound of the present invention may be administered in combination with existing therapeutic agents.

In the present invention, the “administration” means providing a predetermined substance to a patient in any suitable method, and the compound of the present invention may be administered through any general route so long as the compound may reach a target tissue. The administration may be intraperitoneal administration, intravenous administration, intramuscular administration, subcutaneous administration, intradermal administration, oral administration, topical administration, intranasal administration, intrapulmonary administration, and rectal administration, but is not limited thereto. In addition, the compound of the present invention may be administered by any device capable of moving an active substance to a target cell. Preferred administration methods and formulations are intravenous injections, subcutaneous injections, intradermal injections, intramuscular injections, drop injections, and the like. The injections may be prepared by using aqueous solvents such as a physiological saline solution and a ringer solution, and non-aqueous solvents such as vegetable oils, higher fatty acid esters (e.g., ethyl oleate, etc.), and alcohols (e.g., ethanol, benzyl alcohol, propylene glycol, glycerin, or the like). The injections may include pharmaceutical carriers, such as a stabilizer for the prevention of degeneration (e.g., ascorbic acid, sodium hydrogen sulfite, sodium pyrosulfite, BHA, tocopherol, EDTA, etc.), an emulsifier, a buffer for pH control, and a preservative to inhibit microbial growth (e.g., phenyl mercury nitrate, thimerosal, benzalkonium chloride, phenol, cresol, benzyl alcohol, etc.).

In the present invention, the term “therapeutically effective dose” used in combination with an active ingredient refers to a methylsulfonamide-based compound represented by Chemical Formula 1 of the present invention, a stereoisomer thereof, a tautomer thereof, or a pharmaceutically acceptable salt thereof, which is effective for preventing or treating a target disease.

Depending on a type of disease to be prevented or treated, the composition of the present invention may further include known drugs used for the prevention or treatment of each known disease as an active ingredient, in addition to the compounds represented by Chemical Formulas of the present invention, stereoisomers thereof, tautomers thereof, or pharmaceutically acceptable salts thereof. For example, when used for the prevention or treatment of cancer diseases, a known anticancer agent may be further included as an active ingredient in addition to the compound represented by Chemical Formula 1, a stereoisomer thereof, a tautomer thereof, or a pharmaceutically acceptable salt thereof, and may be used in combination with other treatments known for the treatment of these diseases. Other treatments include chemotherapy, radiation therapy, hormone therapy, bone marrow transplantation, stem-cell replacement therapy, other biological therapies, immunotherapy, and the like, but are not limited thereto.

Examples of the anticancer agent which may be included in the composition of the present invention include mechlorethamine, chlorambucil, phenylalanine, mustard, cyclophosphamide, ifosfamide, carmustine (BCNU), lomustine (CCNU), streptozotocin, busulfan, thiotepa, cisplatin, and carboplatin as DNA alkylating agents; dactinomycin (actinomycin D), doxorubicin (adriamycin), daunorubicin, idarubicin, mitoxantrone, plicamycin, mitomycin C, and bleomycin as anti-cancer antibiotics; and vincristine, vinblastine, paclitaxel, docetaxel, etoposide, teniposide, topotecan, and iridotecan as plant alkaloids, and the like, but are not limited thereto.

According to an aspect of the present invention, the present invention provides a composition for inhibiting the activity of CSE1L containing the compound represented by Chemical Formula 1 or a pharmaceutically acceptable salt thereof as an active ingredient. The composition for inhibiting the CSE1L activity of the present invention inhibits the function of CSE1L by selectively binding the compound represented by Chemical Formula 1 contained as an active ingredient to CSE1L.

The composition for inhibiting the CSE1L function of the present invention also inhibits intracellular nuclear transport. An aspect of the present invention provides a method for inhibiting intracellular nuclear transport, comprising treating the composition for inhibiting the CSE1L function to isolated or non-isolated cells. The term “intracellular nuclear transport” of the present invention refers to a selective protein transport during substance exchange between the nucleus and the cytoplasm in eukaryotic cells.

The composition for inhibiting the CSE1L function of the present invention inhibits movement and/or invasion of cancer cells. One aspect of the present invention provides a method for inhibiting movement and/or invasion of cancer cells, comprising treating the composition for inhibiting the CSE1L function to isolated or non-isolated cancer cells, such as solid cancer.

Hereinafter, Synthesis Examples and Examples of compounds of the present invention will be described in detail. However, the following Examples are just illustrative of the present invention, and the contents of the present invention are not limited to the following Examples.

### SYNTHESIS EXAMPLE

Compounds according to the present invention were purchased or synthesized and used. For example, Compound 1-1 (2-[N-methylsulfonyl-4-(trichloromethyl)anilino]acetamide) was purchased from ChemBride (MA, USA) or prepared according to Reaction Formula 1 or 2 below. The following Reaction Formula was only an exemplary method for preparing the compound of the present invention, and the method for preparing the compound of the present invention is not limited thereto, but may be performed using or appropriately modifying a method known in the related art.

In Reaction Formulas 1 and 2 below, R₃ and R₇ were the same as defined above.

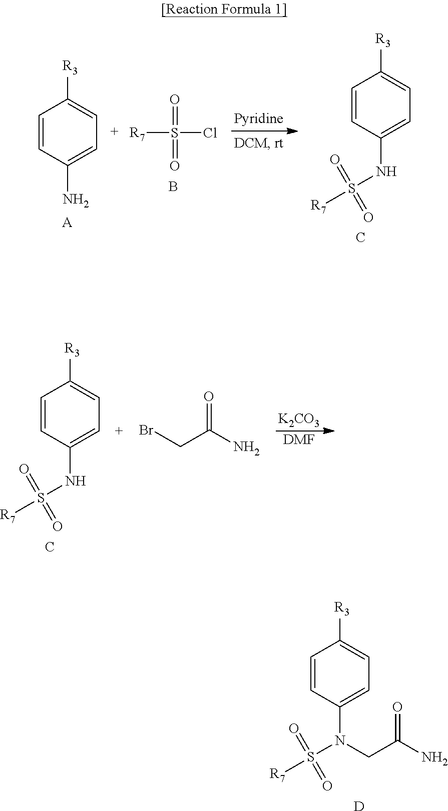

### 1-1) Synthesis Example of Substance C

A starting substance A (100 mg, 0.62 mmol), a substance B (78 mg, 0.053 mmol), and pyridine (147 mg, 1.86 mmol) were dissolved in methylene chloride (0.48 M), and then stirred at room temperature for about 12 hours. After the completion of the reaction was confirmed through TLC, the mixture was extracted with methylene chloride and washed using 1 M HCl and brine. After concentrating the solvent, the mixture was recrystallized under MC/Hex conditions or subjected to column chromatography and then proceed to the next step.

### 1-2) Synthesis Example of Substance D

The synthesized substance C (48 mg, 0.2 mmol), 2-bromoacetamide (83 mg, 0.6 mmol), and K₂CO₃ (55 mg, 0.4 mmol) were added dropwise with DMF, and then stirred at room temperature for about 24 hours. After completion of the reaction, the mixture was extracted with EA and water and washed with brine. After concentrating the solvent, the mixture was recrystallized under EA/Hex conditions or subjected to column chromatography to obtain a final compound D.

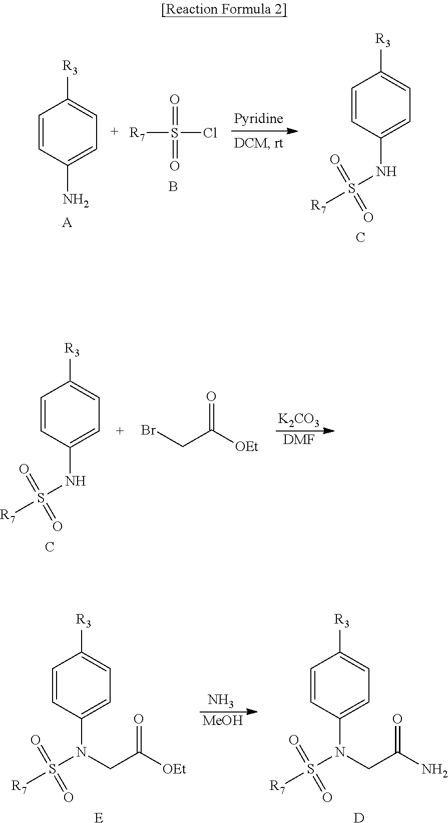

### 1-1) Synthesis Example of Substance C

A substance C was synthesized by applying the same method as synthesized in Reaction Formula 1 above.

### 1-2) Synthesis Example of Substance E

The synthesized substance C (100 mg, 0.42 mmol), 2-ethylbromoacetate (140 mg, 0.83 mmol), and K₂CO₃ (174 mg, 1.26 mmol) were added dropwise with DMF (0.17 M), and then stirred at room temperature for about 24 hours. After completion of the reaction, the mixture was extracted with EA and water and washed with brine. After concentrating the solvent, the mixture was recrystallized under EA/Hex conditions or subjected to column chromatography and then proceed to the next step.

### 1-3) Synthesis Example of Substance D

The synthesized material E (50 mg, 0.15 mmol) and ammonia in methanol solution 7 N (1.7 ml) were added dropwise together, and then stirred at room temperature for about 12 hours. The synthesized substance was formed to white crystals and filtered to obtain a final compound D.

Table 1 below shows yields, and ¹H NMR and ¹³C NMR data of compounds 1-1 to 1-47 of the present invention.

TABLE 1

| Compound | Yield, ¹H NMR and ¹³C NMR |
|---|---|
| 1-1 | Yield 64%; ¹H NMR (400 MHz, DMSO) δ 7.77 (d, J = 8.6 Hz, 2H), 7.67 (d, |
| | J = 8.6 Hz, 2H), 7.53 (s, 1H), 7.22 (s, 1H), 4.36 (s, 2H), 3.17 (s, 3H); ¹³C |
| | NMR (100 MHz, DMSO) δ 169.5, 144.2, 126.7, 126.2, 126.1, 126.1, 125.4, |
| | 122.7, 52.5, 39.2 |
| 1-2 | Yield 67%; ¹H NMR (400 MHz, DMSO) δ 7.72-7.66 (m, 5H), 7.59 (t, J = |
| | 7.2 Hz, 2H), 7.52 (s, 1H), 7.42 (d, J = 8.4 Hz, 2H), 7.16 (s, 1H), 4.31 (s, 2H); |
| | ¹³C NMR (100 MHz, DMSO) δ 168.4, 143.4, 137.7, 133.4, 129.3, 127.3, |
| | 127.2, 125.9, 125.8, 125.2, 122.5, 52.2 |
| 1-3 | Yield 80%; ¹H NMR (400 MHz, DMSO) δ 7.70 (d, J = 8.8 Hz, 2H), 7.56 (s, |
| | 1H), 7.55 (d, J = 8.8 Hz, 2H), 7.40 (t, J = 9.6 Hz, 4H), 7.15 (s, 1H), 4.29 (s, |
| | 2H), 2.38 (s, 3H); ¹³C NMR (100 MHz, DMSO) δ 168.4, 144.0, 143.9, 143.6, |
| | 143.5, 134.8, 129.7, 127.3, 127.1, 126.8, 125.9, 125.8, 125.4, 125.3, 125.2, |
| | 122.6, 52.1, 20.9 |
| 1-4 | Yield 64%; ¹H NMR (400 MHz, DMSO) δ 7.52-7.50 (m, 2H), 7.48-7.45 (m, |
| | 3H), 7.19 (s, 1H), 4.24 (s, 2H), 3.10 (s, 3H); ¹³C NMR (100 MHz, DMSO) δ |
| | 169.7, 139.3, 131.7, 129.1, 129.0, 53.2, 38.8 |
| 1-5 | Yield 93%; ¹H NMR (400 MHz, DMSO) δ 7.72-7.68 (m, 1H), 7.63-7.56 (m, |
| | 4H), 7.43 (s, 1H), 7.41-7.38 (m, 2H), 7.21-7.17 (m, 2H), 7.15 (s, 1H), 4.19 |
| | (s, 2H); ¹³C NMR (100 MHz, DMSO) δ 168.5, 138.5, 137.5, 133.3, 132.0, |
| | 129. 5, 129.2, 128.8, 127.3, 52.8 |
| 1-6 | Yield 88%; ¹H NMR (400 MHz, DMSO) δ 7.50 (d, J = 8.0 Hz, 2H), 7.41 (s, |
| | 1H), 7.40-7.37 (m, 4H), 7.21-7.17 (m, 2H), 7.14 (s, 1H), 4.16 (s, 2H), 2.39 |
| | (s, 3H); ¹³C NMR (100 MHz, DMSO) δ 168.5, 143.7, 138.6, 134.7, 131.9, |
| | 129.7, 129.4, 128.7, 127.3, 52.7, 39.7, 21.0 |
| 1-7 | Yield 84%; ¹H NMR (400 MHz, DMSO) δ 7.86 (s, 1H), 7.80 (d, J = 7.2 Hz, |
| | 1H), 7.69-7.63 (m, 2H), 7.52 (s, 1H), 7.22 (s, 1H), 4.33 (s, 2H), 3.13 (s, 3H); |
| | ¹³C NMR (100 MHz, DMSO) δ 169.6, 141.2, 131.0, 130.3, 129.9, 129.6, |
| | 125.1, 123.9, 123.8, 123.8, 123.7, 122.4, 53.0 |
| 1-8 | Yield 90%; ¹H NMR (400 MHz, DMSO) δ 7.73-7.69 (m, 1H), 7.67-7.60 (m, |
| | 4H), 7.58-7.55 (m, 3H), 7.50 (s, 1H), 7.45 (d, J = 7.6 Hz, 1H), 7.17 (s, 1H), |
| | 4.28 (s, 2H); ¹³C NMR (100 MHz, DMSO) δ 168.5, 140.4, 137.4, 133.4, |
| | 131.4, 130.1, 129.6, 129.3, 127.3, 124.9, 124.7, 124.6, 124.2, 124.1, 122.2, |
| | 52.6, 38.9 |
| 1-9 | Yield 89%; ¹H NMR (400 MHz, DMSO) δ 7.65 (d, J = 8.4 Hz, 1H), 7.58 (s, |
| | 1H), 7.55 (d, J = 7.6 Hz, 1H), 7.49 (d, J = 8.4 Hz, 3H), 7.44 (d, J = 8.4 Hz, |
| | 1H), 7.39 (d, J = 8.4 Hz, 2H), 7.16 (s, 1H), 4.24 (s, 2H), 2.39 (s, 3H); ¹³C |
| | NMR (100 MHz, DMSO) δ 168.5, 143.9, 140.5, 134.5, 131.1, 130.0, 129.7, |
| | 129.5, 129.2, 127.3, 124.6, 124.0, 122.3, 52.5, 21.0 |
| 1-10 | Yield 38%; ¹H NMR (400 MHz, DMSO) δ 7.58 (t, J = 2.0 Hz, 1H), 7.48- |
| | 7.37 (m, 4H), 7.21 (s, 1H), 4.27 (s, 2H), 3.12 (s, 3H); ¹³C NMR (100 MHz, |
| | DMSO) δ 169.6, 141.8, 133.0, 130.6, 127.1, 127.0, 125.6, 53.0 |
| 1-11 | Yield 80%; ¹H NMR (400 MHz, DMSO) δ 7.73-7.69 (m, 1H), 7.65-7.57 (m, |
| | 4H), 7.44 (s, 1H), 7.37-7.34 (m, 2H), 7.31 (t, J = 2.0 Hz, 1H), 7.15 (s, 1H), |
| | 7.14-7.09 (m, 1H), 4.22 (s, 2H); ¹³C NMR (100 MHz, DMSO) δ 168.5, |
| | 141.0, 137.5, 133.4, 132.7, 130.3, 129.2, 127.8, 127.5, 127.3, 125.9, 52.7 |
| 1-12 | Yield 98%; ¹H NMR (400 MHz, DMSO) δ 7.51 (d, J = 8.0 Hz, 2H), 7.44 (s, |
| | 1H), 7.39 (d, J = 8.0 Hz, 2H), 7.37-7.31 (m, 3H), 7.16 (s, 1H), 7.13-7.0 (m, |
| | 1H), 4.19 (s, 2H), 2.39 (s, 3H); ¹³C NMR (100 MHz, DMSO) δ 168.6, |
| | 143.9, 141.1, 140.2, 134.6, 132.7, 130.3, 130.7, 127.7, 127.4, 125.7, 52.6, |
| | 21.0 |
| 1-13 | Yield 41%; ¹H NMR (400 MHz, DMSO) δ 7.81 (t, J = 8.8 Hz, 1H), 7.57 (d, |
| | J = 13.2 Hz, 2H), 7.45 (d, J = 8.8 Hz, 1H), 7.29 (s, 1H), 4.42 (s, 2H), 3.23 (s, |
| | 3H); ¹³C NMR (100 MHz, DMSO) δ 169.3, 160.1, 157.5, 146.4, 146.3, |
| | 127.8, 123.9, 121.2, 120.4, 120.3, 113.6, 113.5, 113.3, 113.2, 112.8, 112.6, |
| | 54.9, 52.0 |
| 1-14 | Yield 33%; ¹H NMR (400 MHz, DMSO) δ 7.89 (d, J = 8.8 Hz, 1H), 7.77 (d, |
| | J = 2.0 Hz, 1H), 7.61 (dd, J = 9.2, 1.6 Hz, 2H), 7.28 (s, 1H), 4.41 (s, 2H), |
| | 3.21 (s, 3H); ¹³C NMR (100 MHz, DMSO) δ 169.3, 145.3, 131.1, 128.6, |
| | 128.5, 127.1, 124.1, 123.9, 123.5, 121.4, 52.1 |
| 1-15 | Yield 31%; ¹H NMR (400 MHz, DMSO) δ 7.91 (t, J = 12.4 Hz, 1H), 7.83 |
| | (dd, J = 10.8, 2.0 Hz, 1H), 7.65 (d, J = 8.0 Hz, 1H), 7.44 (s, 1H), 7.19 (s, 1H), |
| | 4.27 (s, 2H), 3.23 (s, 3H); ¹³C NMR (100 MHz, DMSO) δ 169.1, 160.0, |
| | 157.5, 132.9, 131.6, 131.5, 121.8, 114.4, 114.2, 52.3 |
| 1-16 | Yield 44%; ¹H NMR (400 MHz, DMSO) δ 8.04 (d, J = 1.6 Hz, 1H), 7.97 (d, |
| | J = 8.4 Hz, 1H), 7.80 (dd, J = 8.0, 1.6 Hz, 1H), 7.44 (s, 1H), 7.17 (s, 1H), |
| | 4.24 (s, 2H), 3.27 (s, 3H); ¹³C NMR (100 MHz, DMSO) δ 169.3, 140.9, |
| | 135.1, 133.8, 130.4, 130.0, 127.3, 125.0, 124.4, 121.7, 52.0, 41.3 |
| 1-17 | Yield 87%; ¹H NMR (400 MHz, DMSO) δ 7.41 (s, 1H), 7.37 (d, J = 8.4 Hz, |
| | 2H), 7.20 (d, J = 8.4 Hz, 2H), 7.17 (s, 1H), 4.18 (s, 2H), 3.07 (s, 3H), 2.30 |
| | (s, 3H); ¹³C NMR (100 MHz, DMSO) δ 169.9, 137.8, 137.0, 129.6, 127.6, |
| | 53.6, 20.5 |
| 1-18 | Yield 36%; ¹H NMR (400 MHz, DMSO) δ 7.64-7.60 (m, 2H), 7.49 (s, 1H), |
| | 7.42 (d, J = 7.6 Hz, 2H), 7.21 (s, 1H), 4.26 (s, 2H), 3.12 (s, 3H); ¹³C NMR |
| | (100 MHz, DMSO) δ 144.3, 137.6, 123.9, 122.2, 121.4, 120.9, 118.8, 116.3, |
| | 39.4 |
| 1-19 | Yield 60%; ¹H NMR (400 MHz, DMSO) δ 8.28 (d, J = 8.4 Hz, 1H), 8.20 (t, |
| | J = 4.0 Hz, 2H), 2.09 (d, J = 8.4 Hz, 1H), 7.67 (m, 4H), 7.52 (t, J = 8.8 Hz, |
| | 1H), 7.48 (s, 1H), 7.41 (d, J = 8.4 Hz, 2H), 7.17 (s, 1H), 4.42 (s, 2H); ¹³C |
| | NMR (100 MHz, DMSO) δ 168.4, 143.5, 134.8, 139.9, 133.6, 129.8, 129.0, |
| | 127.9, 127.7, 127.3, 127.2, 127.0, 125.8, 124.6, 124.2, 52.1 |
| 1-20 | Yield 91%; ¹H NMR (400 MHz, DMSO) δ 8.50 (d, J = 8.8 Hz, 1H), 8.21 (d, |
| | J = 7.2 Hz, 1H), 7.86 (d, J = 8.8 Hz, 1H), 7.66-7.60 (m, 3H), 7.50 (s, 1H), |
| | 7.45-7.41 (m, 3H), 7.23 (d, J = 7.2 Hz, 1H), 7.20 (s, 1H), 4.44 (s, 2H), 2.89 |
| | (s, 6H); ¹³C NMR (100 MHz, DMSO) δ 168.6, 162.3, 151.4, 134.7, 134.3, |
| | 130.5, 129.6, 129.2, 129.1, 128.1, 127.0, 125.9, 125.8, 123.7, 118.6, 115.3, |
| | 79.2, 52.0, 45.0, 35.9, 30.8, 29.6 |
| 1-21 | Yield 27%; ¹H NMR (400 MHz, CDCl3) δ 9.09 (dd, J = 4.4, 1.6 Hz, 1H), |
| | 8.38 (dd, J = 8.2, 2.0 Hz, 1H), 8.30 (dd, J = 8.2, 2.0 Hz, 1H), 8.08 (dd, J = |
| | 8.2, 1.6 Hz, 1H), 7.64-7.57 (m, 2H), 7.41 (d, J = 8.8 Hz, 3H), 7.24 (s, 1H), |
| | 4.94 (s, 2H); ¹³C NMR (100 MHz, DMSO) δ 169.7, 151.5, 144.0, 142.8, |
| | 137.1, 136.2, 134.6, 132.9, 128.6, 126.2, 125.8, 125.7, 125.6, 125.3, 122.7, |
| | 122.6, 53.8 |
| 1-23 | Yield 53%; ¹H NMR (400 MHz, DMSO) δ 7.90 (d, J = 8.8 Hz, 2H), 7.75 (t, |
| | J = 7.6 Hz, 6H), 7.54-7.45 (m, 6H), 7.19 (s, 1H), 4.34 (s, 2H); ¹³C NMR (100 |
| | MHz, DMSO) δ 169.0, 142.2, 144.0, 138.5, 136.9, 129.6, 129.1, 128.5, |
| | 128.1, 127.8, 127.5, 127.2, 126.4, 126.4, 125.7, 23.0, 79.6, 52.8 |
| 1-24 | Yield 52%; ¹H NMR (400 MHz, DMSO) δ 7.89 (d, J = 8.4 Hz, 2H), 7.83- |
| | 7.80 (m, 2H), 7.74-7.72 (m, 4H), 7.51 (s, 1H), 7.47 (d, J = 8.4 Hz, 2H), 7.35 |
| | (t, J = 8.8 Hz, 2H), 7.19 (s, 1H), 4.33 (s, 2H); ¹³C NMR (100 MHz, DMSO) |
| | δ 168.5, 163.9, 161.4, 143.6, 143.5, 136.4, 134.5, 129.3, 129.2, 128.0, 127.4, |
| | 127.3, 126.0, 122.6, 116.1, 115.9, 52.3 |
| 1-25 | Yield 11%; ¹H NMR (400 MHz, CDCl₃) δ 8.58 (s, 1H), 7.44 (d, J = 8.2 Hz, |
| | 2H), 7.33-7.29 (m, 2H), 7.25-7.21 (m, 3H), 6.64 (d, J = 8.2 Hz, 2H), 4.80 (s, |
| | 1H), 4.20 (s, 2H), 3.02-2.91 (m, 4H); ¹³C NMR (100 MHz, CDCl₃) δ 149.1, |
| | 139.9, 128.8, 128.6, 128.5, 127.0, 126.7, 11.5, 77.3, 49.0, 39.1, 30.3 |
| 1-26 | Yield 9%; ¹H NMR (400 MHz, CDCl₃) δ 8.44 (s, 1H), 7.45 (d, J = 8.6 Hz, |
| | 2H), 7.32-7.29 (m, 2H), 7.24-7.21 (m, 3H), 6.64 (d, J = 8.6 Hz, 2H), 4.80 (s, |
| | 1H), 4.20 (s, 2H), 3.02-2.91 (m, 4H), 1.58 (s, 2H); ¹³C NMR (100 MHz, |
| | CDCl₃) δ 172.4, 171.9, 149.2, 139.9, 128.8, 128.5, 127.0, 126.9, 126.7, |
| | 126.1, 123.4, 112.6, 77.3, 49.0, 39.1, 30.3 |
| 1-27 | Yield 91%; ¹H NMR (400 MHz, CDCl₃) δ 7.65 (d, J = 8.8 Hz, 2H), 7.66 (d, |
| | J = 8.8 Hz, 2H), 4.38 (s, 2H), 3.14 (s, 3H), 1.50 (s, 9H); ¹³C NMR (100 MHz, |
| | CDCl₃) δ 169.0, 143.7, 129.7, 126.9, 126.8, 126.7, 125.2, 122.5, 83.1, 53.5, |
| | 40.3, 28.1 |
| 1-28 | Yield 84%; ¹H NMR (400 MHz, CDCl₃) δ 7.68 (d, J = 8.8 Hz, 2H) 7.61 (d, |
| | J = 8.8 Hz, 2H), 4.58 (s, 2H), 3.12 (s, 3H); 13C NMR (100 MHz, CDCl₃) δ |
| | 175.1, 143.1, 130.3, 130.0, 127.0, 125.0, 122.3, 52.4, 40.1 |
| 1-29 | Yield 42%; ¹H NMR (400 MHz, CDCl₃) δ 7.68-7.62 (m, 4H), 6.03 (s, 1H), |
| | 4.36 (s, 2H), 3.07 (s, 3H), 2.86 (d, J = 4.8 Hz, 3H); ¹³C NMR (100 MHz, |
| | CDCl₃) δ 168.5, 143.3, 130.1, 129.8, 127.0, 125.1, 122.4, 54.5, 38.8, 29.8, |
| | 26.6 |
| 1-30 | Yield 50%; ¹H NMR (400 MHz, DMSO) δ 10.70 (s, 1H), 8.98 (s, 1H), 7.78 |
| | (d, J = 8.8 Hz, 2H), 7.66 (d, J = 8.8 Hz, 2H), 4.30 (s, 2H), 3.19 (s, 3H); ¹³C |
| | NMR (100 MHz, CD₃OD) δ 143.3, 128.6, 127.6, 127.6, 127.3, 127.1, 127.0, |
| | 119.9, 40.2, 39.7 |
| 1-31 | Yield 45%; ¹H NMR (400 MHz, DMSO) δ 8.22-8.16 (m, 4H), 8.00 (s, 1H), |
| | 4.92 (s, 2H), 4.46 (s, 2H), 3.60 (s, 3H); ¹³C NMR (100 MHz, CDCl₃) δ 168.7, |
| | 142.2, 130.3, 130.0, 127.1, 127.0, 127.0, 126.8, 126.7, 125.0, 122.3, 53.2, |
| | 52.9, 40.4, 38.9 |
| 1-32 | Yield 84%; ¹H NMR (400 MHz, DMSO) δ 7.76 (d, J = 8.6 Hz, 2H), 7.67 (d, |
| | J = 8.6 Hz, 2H), 7.50 (s, 1H), 7.20 (s, 1H), 4.37 (s, 2H), 3.34 (d, J = 7.6 Hz, |
| | 1H), 3.31 (d, J = 7.4 Hz, 1H), 1.23 (d, J = 7.6 Hz, 3H); ¹³C NMR (100 MHz, |
| | DMSO) δ 169.4, 144.2, 126.7, 126.5, 126.2, 126.1, 125.4, 125.7, 52.5, 46.4, |
| | 7.6 |
| 1-33 | Yield 50%; 1H NMR (400 MHz, DMSO) δ 7.74 (d, J = 8.6 Hz, 2H), 7.69 |
| | (d, J = 8.6 Hz, 2H), 7.44 (s, 1H), 7.15 (s, 1H), 4.39 (s, 2H), 1.26 (d, J = 6.4 |
| | Hz, 6H); ¹³C NMR (100 MHz, DMSO) δ 169.2, 144.5, 126.7, 126.6, 126.3, |
| | 126.0, 125.9, 125.4, 122.7, 53.7, 52.7, 16.4 |
| 1-34 | Yield 61%; ¹H NMR (400 MHz, DMSO) δ 7.73 (d, J = 9.0 Hz, 2H), 7.71 (d, |
| | J = 9.0 Hz, 2H), 7.50 (s, 1H), 7.20 (s, 1H), 4.36 (s, 2H), 0.99-0.94 (m, 2H), |
| | 0.84-0.80 (m, 2H); ¹³C NMR (100 MHz, DMSO) δ 169.4, 144.4, 127.5, |
| | 127.2, 126.8, 126.0, 125.9, 125.4, 122.7, 53.0, 29.1, 5.1 |
| 1-35 | Yield 21%; ¹H NMR (400 MHz, CDCl3) δ 7.61 (d, J = 8.8 Hz, 2H), 7.30 (d, |
| | J = 8.2 Hz, 2H), 7.15 (s, 1H), 7.03 (d, J = 8.2 Hz, 1H), 6.92 (d, J = 8.8 Hz, |
| | 1H), 6.46 (s, 1H), 5.48 (s, 1H), 4.34-4.29 (m, 4H), 4.21 (s, 2H); ¹³C NMR |
| | (100 MHz, CDCl₃) δ 169.8, 148.4, 143.8, 142.9, 128.6, 127.6, 126.7, 121.7, |
| | 118.0, 117.4, 77.3, 64.7, 64.2, 54.1 |
| 1-36 | Yield 37%; ¹H NMR (400 MHz, CDCl₃) δ 7.61 (d, J = 8.8 Hz, 2H), 7.51 (d, |
| | J = 8.8 Hz, 2H), 7.43 (t, J = 8.0 Hz, 2H), 7.31 (d, J = 8.0 Hz, 2H), 7.27-7.24 |
| | (m, 1H), 7.08 (d, J = 8.8 Hz, 2H), 7.00 (d, J = 8.8 Hz, 2H), 6.46 (s, 1H), 5.63 |
| | (s, 1H), 4.22 (s, 2H); ¹³C NMR (100 MHz, CDCl₃) δ 169.7 162 8 154 6 142 |
| | 9 130.4 130.2 129.6 127.6 126.8 126.7 126.7 126.7 125.5 120.6 117.5 54.1 |
| 1-37 | Yield 24%; ¹H NMR (400 MHz, DMSO) δ 9.31(d, J = 5.0 Hz, 1H), 9.15 (s, |
| | 1H), 8.16 (s, 1H), 8.12 (d, J = 5.0 Hz, 1H), 7.74 (d, J = 8.2 Hz, 2H), 7.57 (d, |
| | J = 8.2 Hz, 3H), 7.28 (s, 1H), 4.41 (s, 2H); ¹³C NMR (100 MHz, DMSO) δ |
| | 169.0, 163.5, 154.8, 143.5, 142.2, 132.5, 127.1, 126.3, 126.3, 123.3, 116.7, |
| | 52.8 |
| 1-38 | Yield 17%; ¹H NMR (400 MHz, DMSO) δ 7.87 (d, J = 8.4 Hz, 2H), 7.74- |
| | 7.70 (m, 4H), 7.66 (d, J = 8.2 Hz, 2H), 9.61 (s, 1H), 7.46 (d, J = 8.4 Hz, 2H), |
| | 7.21-7.19 (m, 2H), 4.33 (s, 2H); ¹³C NMR (100 MHz, CD₃OD) δ 129.7, |
| | 129.4, 128.1, 127.2, 127.1, 126.9, 126.6, 79.4, 30.7 |
| 1-39 | Yield 22%; 1H NMR (400 MHz, DMSO) δ 8.96 (s, 1H), 8.04 (d, J = 8.8 Hz, |
| | 2H), 8.00 (s, 1H), 7.79 (d, J = 8.4 Hz, 2H), 7.73 (d, J = 8.8 Hz, 2H), 7.50 (s, |
| | 1H), 7.46 (d, J = 8.4 Hz, 2H), 7.19 (s, 1H), 4.33 (s, 2H); ¹³C NMR (100 MHz, |
| | DMSO) δ 168.6, 143.4, 142.6, 142.2, 135.2, 129.3, 128.8, 127.7, 126.1, |
| | 126.1, 118.4, 96.3, 52.5 |
| 1-40 | Yield 90%; ¹H NMR (400 MHz, DMSO) δ 8.79 (d, J = 1.8 Hz, 1H), 8.00 (d, |
| | J = 7.2 Hz, 2H), 7.92 (d, J = 1.8 Hz, 1H), 7.76 (t, J = 8.4 Hz, 1H), 7.70-7.64 |
| | (m, 4H), 7.55 (s, 1H), 7.47 (d, J = 8.4 Hz, 2H), 7.23 (s, 1H), 4.35 (s, 2H); ¹³C |
| | NMR (100 MHz, DMSO) δ 168.3, 142.7, 141.2, 141.2, 140.3, 139.2, 134.2, |
| | 130.6, 129.8, 127.9, 127.3, 126.1, 126.1, 52.9 |
| 1-41 | Yield 10%; ¹H NMR (400 MHz, DMSO) δ 8.62 (s, 1H), 8.30 (dd, J = 4.4, |
| | 1.2 Hz, 1H), 7.78-7.30 (m, 4H), 7.52 (s, 1H), 7.47 (d, J = 8.8 Hz, 2H), 7.45- |
| | 7.41 (m, 2H), 7.36 (d, J = 8.8 Hz, 1H), 7.20 (s, 1H), 4.34 (s, 2H); ¹³C NMR |
| | (100 MHz, DMSO) δ 168.5, 164.5, 156.8, 145.3, 138.1, 138.0, 134.2, 129.5, |
| | 127.3, 126.1, 126.0, 122.0 112.0, 52.3 |
| 1-42 | Yield 89%; ¹H NMR (400 MHz, CDCl3) δ 7.66 (d, J = 8.4 Hz, 2H), 7.61 |
| | (t, J = 2.0 Hz, 1H), 7.48 (d, J = 2.0 Hz, 2H), 7.35 (d, J = 8.4 Hz, 2H). 6.21 (s, |
| | 1H), 5.57 (s, 1H), 4.28 (s, 2H); ¹³C NMR (100 MHz, CDCl₃) δ 168.9, 142.1, |
| | 139.6, 136.4, 133.8, 128.0, 127.0, 126.2, 77.3, 54.3 |
| 1-43 | Yield 12%; ¹H NMR (400 MHz, CDCl₃) δ 7.88-7.86 (m, 2H), 7.77(d, J = |
| | 8.0 Hz, 1H), 7.64-7.57 (m, 4H), 7.53-7.48 (m, 2H), 3.40 (t, J = 7.6 Hz, 1H), |
| | 7.34 (d, J = 6.8 Hz, 1H), 6.00 (s, 1H), 5.46 (s, 1H), 4.41 (s, 2H), 3.58 (s, 4H); |
| | ¹³C NMR (100 MHz, CDCl₃) δ 134.0, 133.4, 132.5, 131.4, 129.2, 128.2, |
| | 127.0, 127.0, 126.9, 126.8, 126.8, 126.1, 125.7, 122.8, 77.3, 52.7, 26.8 |
| 1-44 | Yield 13%; ¹H NMR (400 MHz, CDCl₃) δ 7.86-7.84 (m, 2H), 7.74-7.72 (m, |
| | 2H), 7.67 (s, 4H), 5.98 (s, 1H), 5.51 (s, 1H), 4.45(s, 2H), 4.16 (t, J = 6.8 Hz, |
| | 2H), 3.63 (t, J = 6.8 Hz, 2H); ¹³C NMR (100 MHz, CDCl₃) δ 169.8, 167.6, |
| | 142.7, 134.4, 131.9, 127.7, 127.2, 127.1, 123.7, 77.3, 54.4, 49.5, 32.3 |
| 1-45 | Yield 10%; ¹H NMR (400 MHz, CDCl₃) δ 7.71-7.66 (m, 4H), 5.69 (s, 1H), |
| | 5.50 (s, 1H), 4.45 (s, 2H), 4.16-4.10 (m, 2H); ¹³C NMR (100 MHz, CDCl₃) |
| | δ 169.7, 142.2, 128.1, 127.3, 127.2, 77.3, 55.0, 54.9, 54.6, 54.3 |
| 1-46 | Yield 72%; ¹H NMR (400 MHz, CDCl₃) δ 7.68 (s, 4H), 5.69 (s, 1H), 5.47 |
| | (s, 1H), 4.42 (s, 2H), 3.51-3.47 (m, 2H), 2.71-2.60 (m, 2H); ¹³C NMR (100 |
| | MHz, CDCl₃) δ 169.8, 142.9, 127.9, 127.2, 127.1, 77.4, 68.8, 54.5, 53.0, |
| | 46.0, 28.9, 28.6 |
| 1-47 | Yield 13%; ¹H NMR (400 MHz, CDCl₃) δ 7.62 (d, J = 8.4 Hz, 2H), 7.49 (d, |
| | J = 8.4 Hz, 2H), 7.41-7.36 (m, 5H), 6.02 (s, 1H), 5.37 (s, 1H), 4.47 (s, 2H), |
| | 4.12 (s, 2H); ¹³C NMR (100 MHz, CDCl₃) δ 169.9, 143.0, 131.0, 129.5, |
| | 129.2, 127.6, 126.9, 126.8, 125.9, 77.4, 58.3, 54.5 |

### <Example 1> Discovery of Novel Methylsulfonamide-Based Compounds of Metastasis-Targeting Protein (CSE1L) Using Molecular Modeling

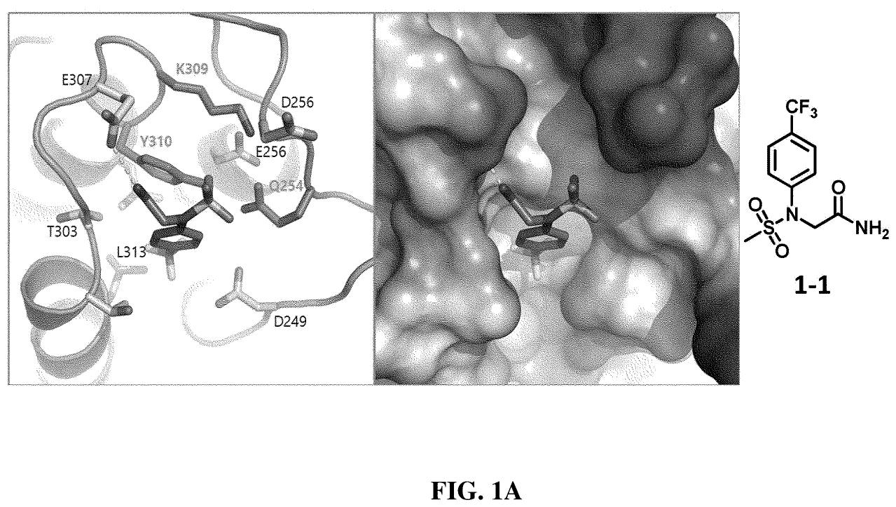

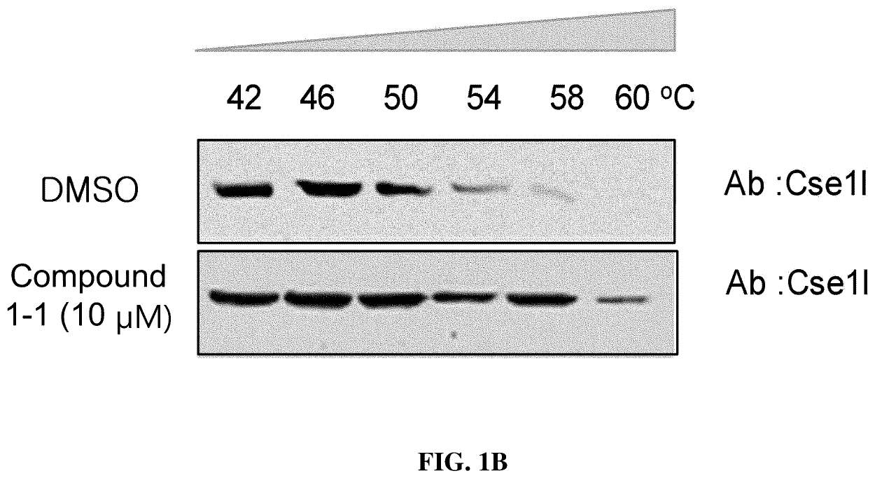

A CSE1L protein was known as nuclear exportin 2, a type of nuclear exportin that played a role in moving importin-α from the intra-nucleus to the cytoplasm as a cargo protein of nuclear transport (Solsbacher, J.; Maurer, P.; Bischoff, F. R.; Schlenstedt, G. Mol. Cell. Biol. 1998, 18, 6805.). In general, importin-α and Ran-GTP, which were cargo proteins, bound to exportin CSE1L in the nucleus and transported to the cytoplasm through nuclear pores, and the obtained CSE1L-bound Ran-GTP protein was rapidly hydrolyzed to Ran GDP to have the form of a cargo free state (a state in which importin-ax was not bound). Through this series of processes, the protein played a role of substance transport. As another function, the CSE1L protein was particularly abundant in cancer cells or tissues, and reported to have roles and functions in metastasis, such as carcinogenesis, cancer cell movement, and cancer cell invasion, but since the exact mechanism has not been reported yet, it was intended to predict the interaction with a novel metastasis-targeting protein (CSE1L) and develop effective substances using molecular modeling tools. Based on a binding site of the previously identified fusarisetin compound and the target protein (CSE1L), a virtual search was conducted using about 1.5 million compound libraries (commercial focused libraries) to predict combinable compounds. As a result, it was confirmed that the methylsulfonamide-based compound as the compound 1-1 of the present invention could bind to the target protein CSE1L (FIG. 1A). A thermal shift assay (TSA) was performed to confirm whether the compound 1-1 discovered in this way bound to the target protein (CSE1L) in cells (FIG. 1B). First, the cells were treated with DMSO and the compound 1-1 (10 vM) and incubated in a CO₂ incubator at 37° C. for 24 hours. Thereafter, the protein was isolated and treated at 42, 46, 50, 54, 59, and 60° C. for 5 minutes using a PCR machine, and then denatured proteins were removed by centrifuge (15,0000 rpm), and the amount of remaining CSE1L was confirmed by Western blot. As a result, it was confirmed that the expression level of the protein treated with the compound 1-1 was not reduced, but continuously maintained through structural stabilization.

### <Example 2> Evaluation of Verifying Cytotoxicity, Cancer Cell Movement and Metastasis-Inhibiting Activity of Compound 1-1

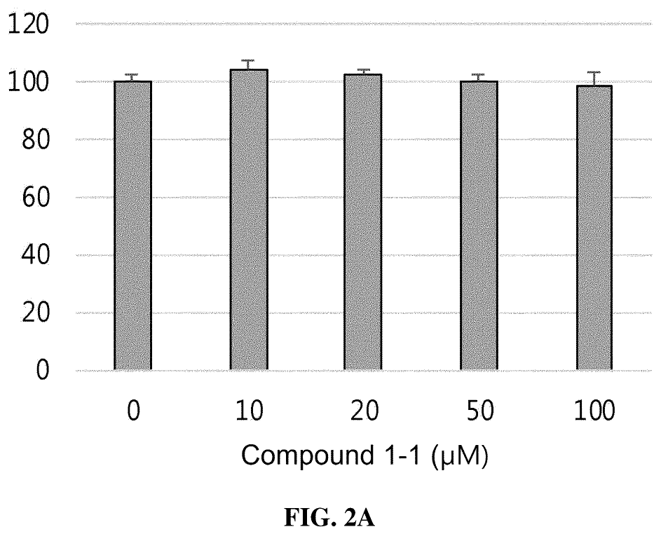

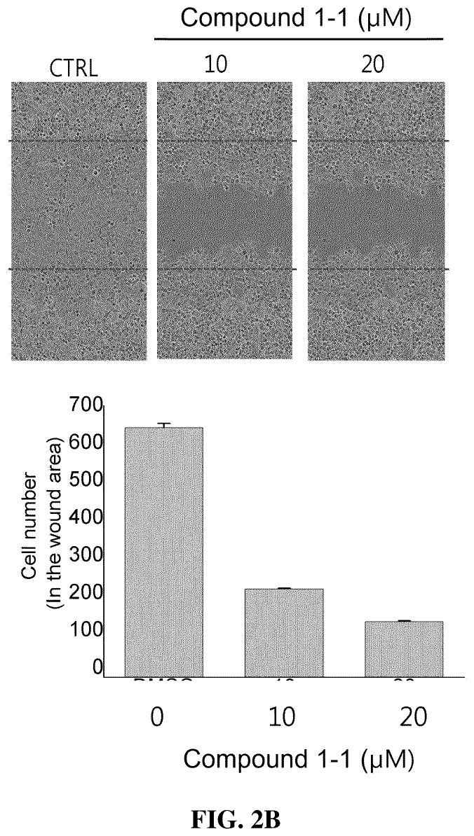

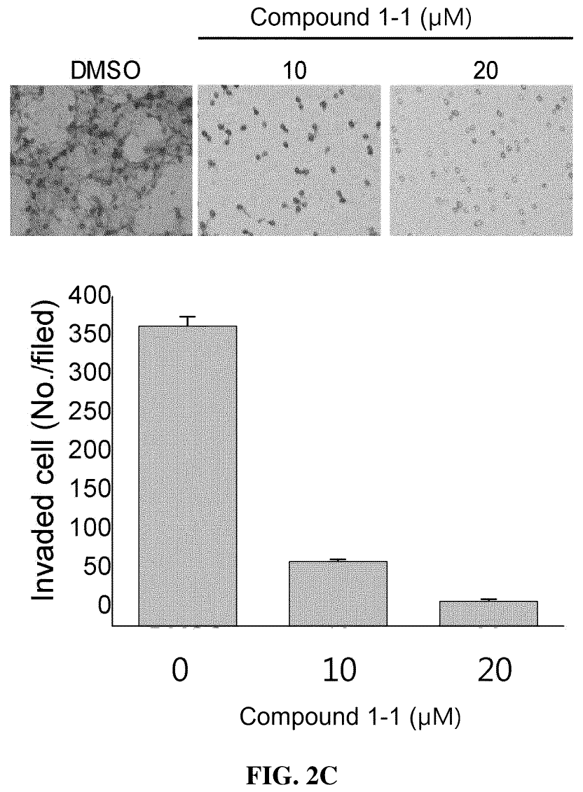

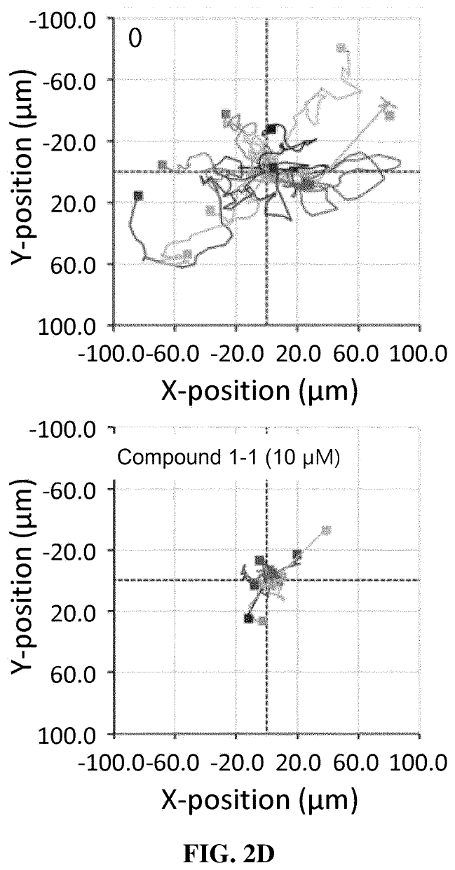

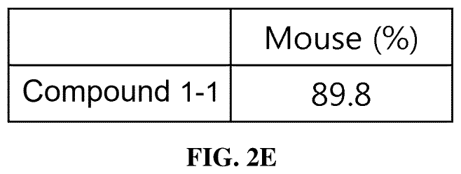

In order to evaluate the cytotoxicity of the compound 1-1 according to the present invention, MTT assay was performed on a human breast cancer cell line (MDA-MB-231). Cells used in the experiment were all purchased from the American Type Culture Collection (ATCC). First, 1×10⁵ MDA-MB-231 cells were divided into 200 vL of 5×10³ cells in a 96-well plate using a DMEM medium added with 10% fetal bovine serum (FBS), 50 mg/ml streptomycin and 50 U/ml penicillin while maintained at 37° C. under 5% CO₂ air, and then incubated in a CO₂ incubator for 24 hours. The purchased compound 1-1 of the present invention was treated at a concentration of 50 vM and incubated for 24 hours under the same culture conditions. After the incubation was completed, the culture medium was removed and 50 vL of 10 mg/mL MTT solution [3-(4,5-dimethylthiazol-2-yl)-2,5-diphenyltetrazolium bromide] (Sigma, St. Louis, MO, USA) was added per well and reacted at 37° C. for 4 hours. Subsequently, a supernatant was removed carefully so as to remove formazan crystals at the bottom, and 50 μL of dimethyl sulfoxide (DMSO) was added per well, and then shaken for 10 minutes, and an optical density (OD) at 590 nm was measured. As a result, it was confirmed that the compound of the present invention had no cytotoxicity at an effective concentration of 50 vM (FIG. 2A). Next, Wound healing assay was performed on MDA-MB-231 cells at concentrations of 10 and 20 μM, which were non-toxic concentrations of the compound 1-1 according to the present invention, to measure movement control ability of cancer cells. First, the MDA-MB-231 cell line was applied to a 12-well plate at 1×10⁵ cells. After incubation for 24 hours, the cell line was wound at a certain area using a yellow tip, washed with PBS, and after 30 minutes, treated with the compound 1-1 (10 and 20 vM) of the present invention for 24 hours, and then the movement of the cancer cell line was observed. As a result, it was confirmed that the cell movement of the cancer cell line treated with the compound 1-1 of the present invention was significantly reduced compared to a control group (FIG. 2B). That is, it may be seen that the compound according to the present invention may effectively inhibit metastasis by reducing the movement of cancer cells. Next, in order to confirm the cancer cell invasion ability of the compound 1-1, a transwell chamber (BioCoat™ Matrigel™ Invasion Chamber, pore size of 8 m, BD Biosciences, Bedford, MA) was used. First, the lower side of a transwell membrane was coated with 50 ml of 0.1% Matrigel. 10% FBS was added to the lower part of a transwell cell culture chamber, 1×10⁵ MDA-MB-231 cell lines and the compound 1-1 (10 and 20 vM) of the present invention were added to a serum-free culture medium on the top of the transwell, and then incubated at 37° C. for 20 hours. Non-moved cells on the top of the filter were removed with a cotton swab, and cells that moved through the filter were stained with crystal violet staining. The number of moved cells was quantified using an optical microscope (×100). As a result, it was confirmed that the number of invaded cancer cells was significantly reduced compared to the control group (FIG. 2C). Next, cell movement was confirmed using a Holomonitor device to determine whether to affect cell movement in real time. As a result, it was confirmed that the methylsulfonamide-based compound inhibited the invasion and movement of cancer cells (FIG. 2D). Next, mouse liver metabolic stability assay was performed to confirm the in vivo metabolic stability of the methylsulfonamide-based compound, and as a result, it was confirmed that 89.8% of the compound had very good metabolic stability (FIG. 2E).

### <Experimental Example 3> Evaluation of Anti-Metastatic Efficacy of Compound 1-1 Using Metastatic Breast Cancer Model

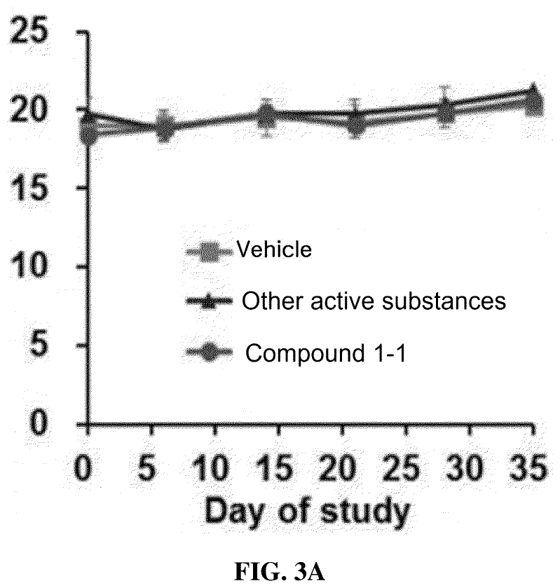

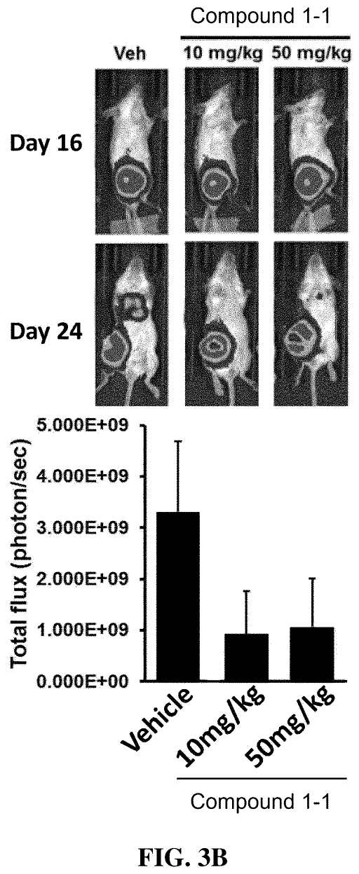

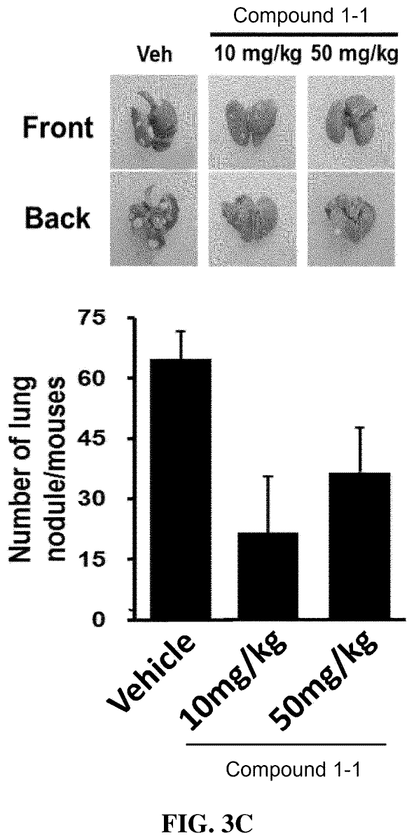

In order to test a metastasis-inhibiting effect of the compound 1-1, which was a CSE1L inhibiting compound, in a mouse model, the metastasis-inhibiting effect was confirmed using an orthotopic xenograft spontaneous metastasis model. First, a method of examining metastasis of cancer formed by subcutaneous transplantation of mouse breast cancer cell line 4T1 (murine breast cancer cell) to other tissues was used. Female 6-week-old nude mice (BALB/c) were used. To make solid cancer, the mouse breast cancer cell line 4T1 (1×10⁵ murine breast cancer cells) was transplanted into the 4th Mammary Fat Pad of the mouse, and from 5 days later, the compound 1-1 was treated at 10 mg/kg and 50 mg/kg every day for 3 weeks, respectively. As a result, it was confirmed that since there was no change in body weight compared to a negative control group, there was no toxicity of the drug (FIG. 3A). In addition, the volume and capacity of the tumors were not suppressed during the drug treatment period (16 to 24 days), but in the control group, significant cancer tissue was formed and metastasized to other organs, such as the lung and heart, whereas the treatment with the CSE1L inhibitor compound 1-1 showed a tendency to inhibit the formation of cancer tissue in other organs (FIG. 3B). In addition, after 25 days, the mice were autopsied to confirm the number of cancer cells metastasizing to the lung. As a result, it was confirmed that the number of cancer cells metastasizing to the lung was significantly reduced in the compound 1-1 treated group compared to the control group (FIG. 3C). Through the evaluation of anti-metastasis efficacy using the metastasis breast cancer model, the metastasis-inhibiting efficacy of the compound 1-1 was confirmed through selective regulation of the CSE1L function by binding to the target protein CSE1L. Accordingly, a novel derivative was synthesized in order to secure a substance showing better activity than these substances.

### <Example 4> Evaluation of Cytotoxicity Analysis for Compounds 1-1 to 1-47

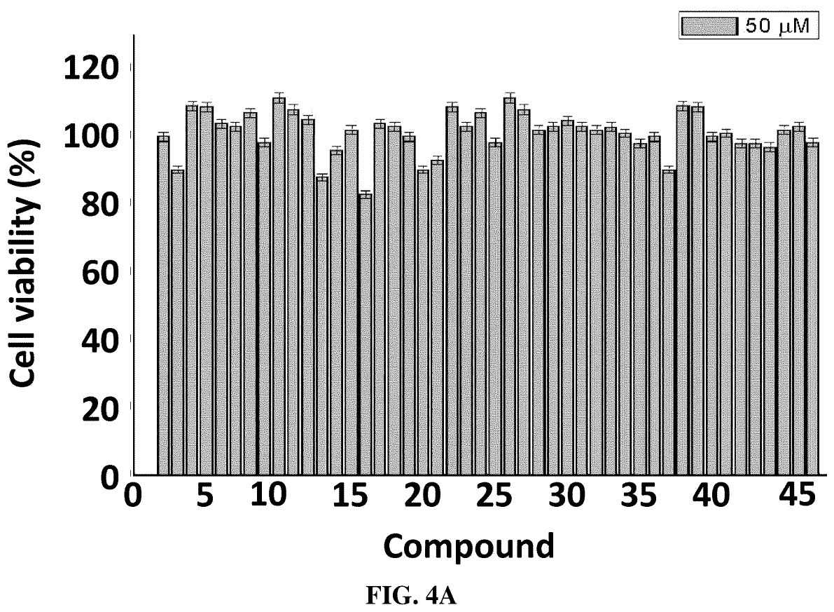

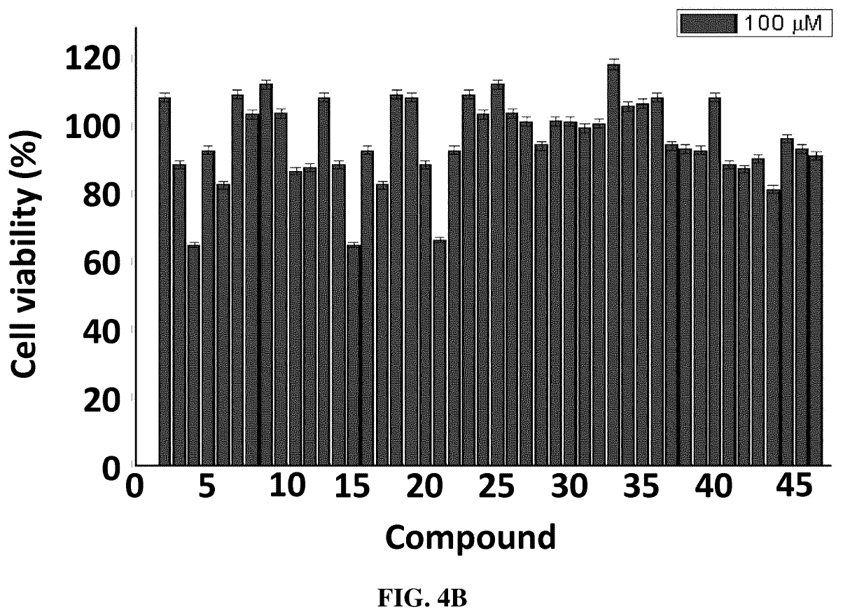

In order to evaluate the cytotoxicity of compounds 1-1 to 1-47 according to the present invention, MTT assay was performed on a human breast cancer cell line (MDA-MB-231). As a result, it was confirmed that the compounds of the present invention had no cytotoxicity at effective concentrations of 50 and 100 vM, except for the 25th substance (FIGS. 4A and 4B).

### <Example 5> Evaluation of Analyzing Movement Inhibition of Cancer Cells for Compounds 1-1 to 1-47

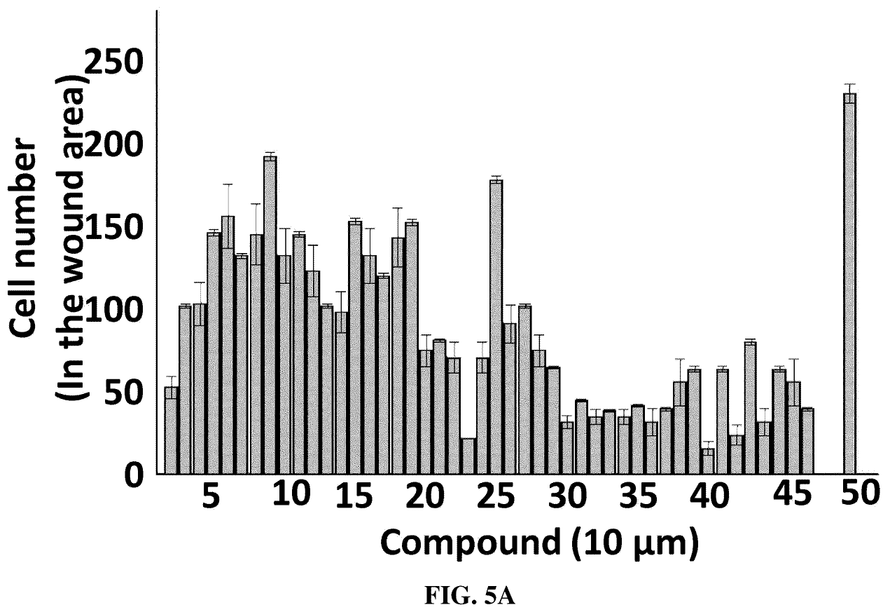

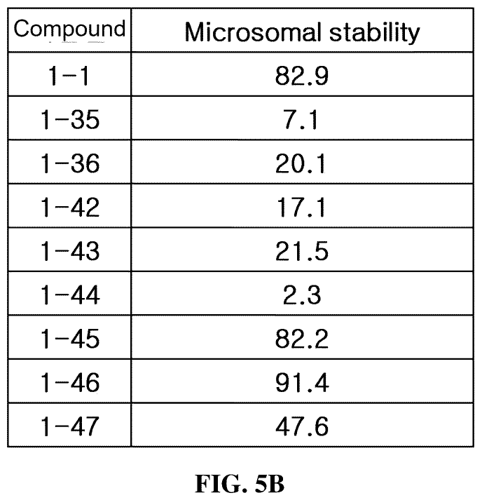

Next, Wound healing assay was performed on MDA-MB-231 cells at concentrations of 10 and 20 μM, which were non-toxic concentrations in the compounds 1-1 to 1-47 according to the present invention, to measure movement control ability of cancer cells. As a result, it was confirmed that the cell movement of the cancer cell lines treated with the compounds 1-1 to 1-47 of the present invention was significantly reduced compared to a control group (FIG. 5A). Next, in order to confirm the in vivo metabolic stability of methylsulfonamide-based derivative compounds of the compounds 1-1, 1-35, 1-36, and 1-42 to 1-47, mouse liver metabolic stability assay was performed, and among them, 82.2% of the compound 1-45 and 91.4% of the compound 1-46 were confirmed to have excellent metabolic stability (FIG. 5B). Accordingly, the compound 1-46 was used in experiments later.

### <Example 6> Evaluation of Analyzing Movement Inhibition of Cancer Cells for Compound 1-46

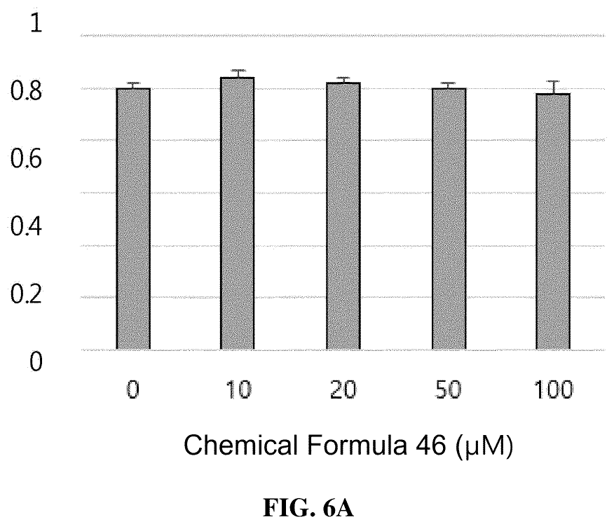

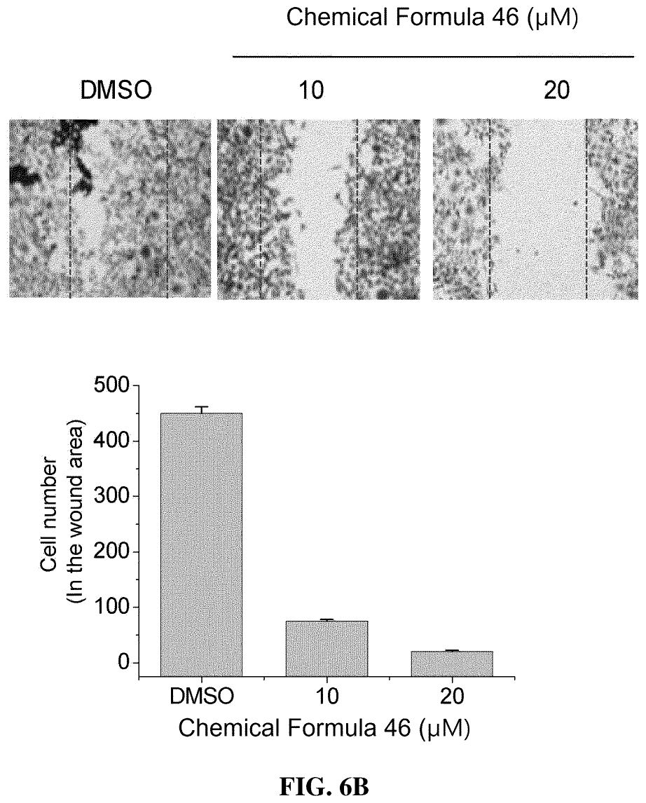

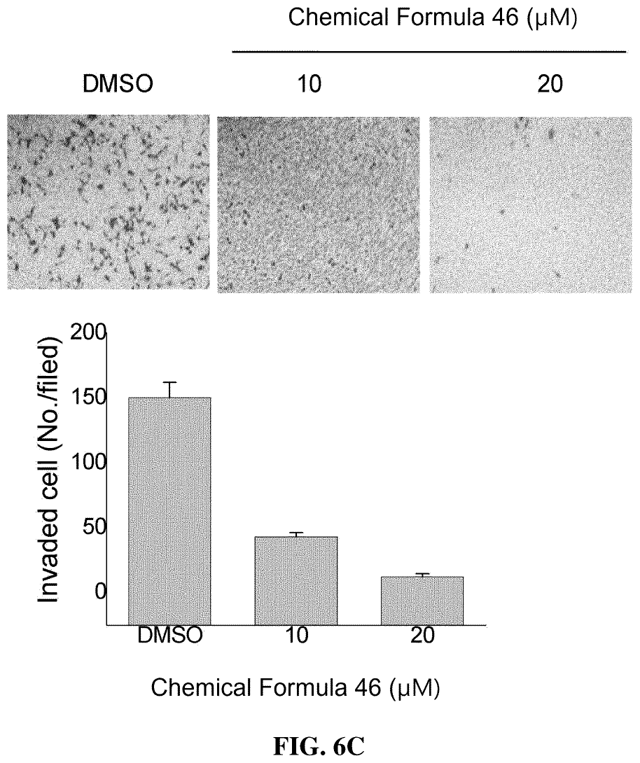

In order to evaluate the cytotoxicity of the compound 1-46 according to the present invention, MTT assay was performed on a human breast cancer cell line (MDA-MB-231). As a result, it was confirmed that the compound of the present invention had no cytotoxicity at effective concentrations of 10 to 100 vM (FIG. 6A). Next, Wound healing assay was performed on MDA-MB-231 cells to measure movement control ability of cancer cells. As a result, it was confirmed that the cell movement of the cancer cell line treated with the compound 1-46 of the present invention was significantly reduced compared to a control group (FIG. 6B). Next, in order to confirm the cancer cell invasion ability of the compound 1-46 (10 and 20 vM), a transwell chamber (BioCoat™ Matrigel™ Invasion Chamber, pore size of 8 m, BD Biosciences, Bedford, MA) was used. As a result, it was confirmed that the number of invaded cancer cells was significantly reduced compared to the control group, similarly to the cell movement experiment (FIG. 6C).

### <Experimental Example 7> Verification of Angiogenesis Inhibition for Compound 1-46

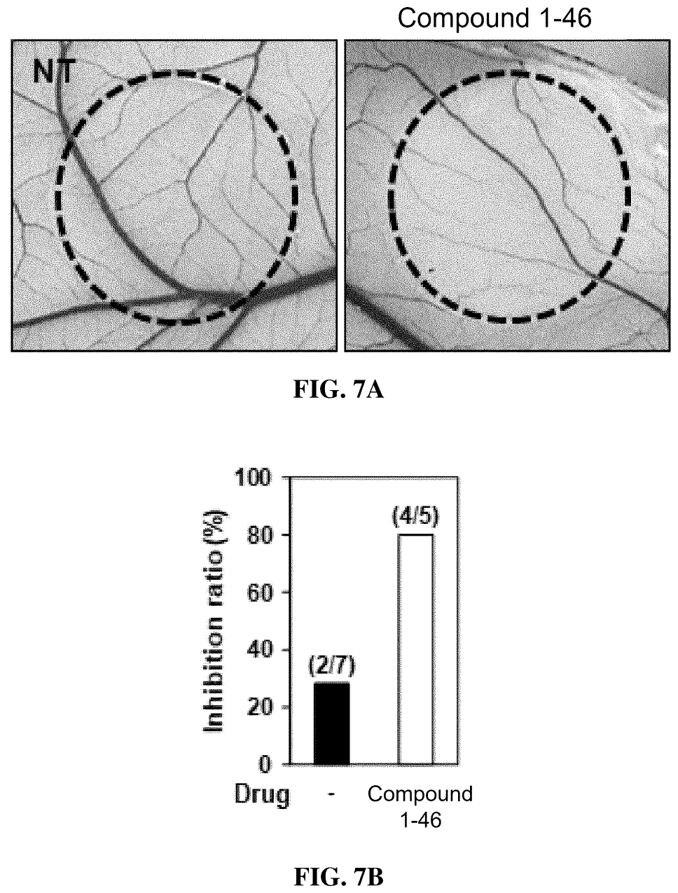

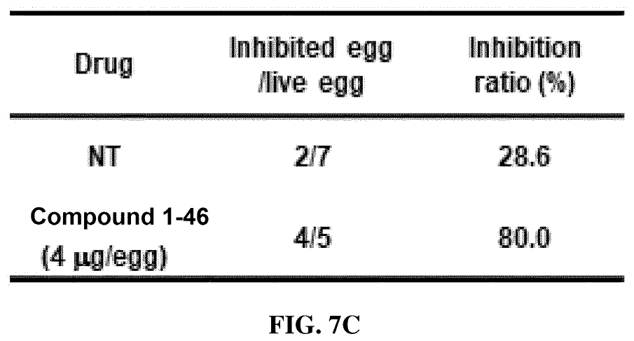

In order to confirm whether angiogenesis was inhibited using the compound 1-46, which had high stability in vivo, among methylsulfonamide-based derivative compounds discovered as CSE1L inhibitors, a chorioallantoin membrane (CAM) was performed. First, the surface of the fertilized viable egg was wiped with 70% ethanol, incubated at 37° C. for 4 days, and 3 ml of albumin of the viable egg was extracted and then treated with the compound 1-46 at a concentration of 4 μg, and then it was observed whether angiogenesis was inhibited. As a result, it was confirmed that the compound 1-46 had excellent angiogenesis inhibitory activity (FIGS. 7A-7C).

### <Preparation Example 1> Preparation of Powders

Methylsulfonamide-based derivative compound of the present invention 0.1 g

| | |
|---|---|
| Lactose | 1.5 g |
| Talc | 0.5 g |

The ingredients were mixed and filled in an airtight bag to prepare powders.

### <Preparation Example 2> Preparation of Tablets

Methylsulfonamide-based derivative compound of the present invention 0.1 g

| | |
|---|---|
| Crystalline cellulose | 1.5 g |
| Magnesium stearate | 0.5 g |

The ingredients were mixed and then tablets were prepared by a direct tableting method.

### <Preparation Example 3> Preparation of Capsules

Methylsulfonamide-based derivative compound of the present invention 0.1 g

| | |
|---|---|
| Corn starch | 5 g |
| Carboxy cellulose | 4.9 g |

The ingredients were mixed to prepare powder, and then the powder was filled in hard capsules according to a general capsule preparation method to prepare capsules.

### <Preparation Example 4> Preparation of Injections

Methylsulfonamide-based derivative compound of the present invention 0.1 g

| | |
|---|---|
| Sterile distilled water for injection | Suitable amount |
| pH adjusting agent | Suitable amount |

The injections were prepared with the ingredient content per 1 ampoule (2 ml) according to a general method for preparing injections.

### <Preparation Example 5> Preparation of Liquids

Methylsulfonamide-based derivative compound of the present invention 0.1 g

| | |
|---|---|
| Isomerized sugar | 10 g |
| Mannitol | 5 g |
| Purified water | Suitable amount |

Each ingredient was added and dissolved to purified water according to a general method for preparing liquids and added with a suitable amount of lemon flavor and then the ingredients were mixed. Thereafter, the mixture was added with purified water to adjust the total volume to 100 ml, and then filled into a brown bottle and sterilized to prepare liquids.

The above description just illustrates the technical spirit of the present invention and various changes and modifications can be made by those skilled in the art to which the present invention pertains without departing from an essential characteristic of the present invention. Therefore, the exemplary examples of the present invention are provided for illustrative purposes only but not intended to limit the present invention. The concept and scope of the present invention are not limited to the exemplary examples. The protective scope of the present invention should be construed based on the following claims, and all the techniques in the equivalent scope thereof should be construed as falling within the scope of the present invention.

1. A compound represented by Chemical Formula 1 below, a stereoisomer thereof, or a pharmaceutically acceptable salt thereof. 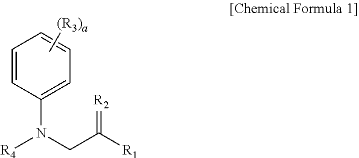
{In Chemical Formula 1,
1) R₁ is OH, NH—R₅ or O—R₆,
2) the R₅ is H, OH, NH₂ or a C₁-C₁₀ alkyl group,
3) the R₆ is a C₁-C₁₀ alkyl group,
4) R₂ is O or absent,
5) R₃ is the same as or different from each other, and is selected from the group consisting of hydrogen; halogen; a C₁-C₁₀ alkyl group; a C₁-C₁₀ alkoxy group; a C₁-C₁₀ alkyl group substituted with fluorine; and a C₁-C₁₀ alkoxy group substituted with fluorine,
6) a is an integer of 0 to 5,
7) R₄ is —SO₂—R₇; or —CO₂—(CH₂)ₘ—R₈,
8) the R₇ is selected from the group consisting of a C₁-C₁₀ alkyl group; a C₁-C₁₀ alkyl group substituted with fluorine; a C₃-C₁₀ cycloalkyl group; a C₆-C₂₄ aryl group; a C₂-C₂₄ heterocyclic group; and —(CH₂)ₙ—R₉,
9) the R₈ and R₉ are each independently a C₆-C₂₄ aryl group; or a C₂-C₂₄ heterocyclic group,
10) m and n are independently integers of 0 to 5,
11) wherein, each of the alkyl group, alkoxy group, cycloalkyl group, aryl group, and heterocyclic group may be further substituted with at least one substituent selected from the group consisting of halogen; a C₁-C₁₀ alkyl group; a C₁-C₁₀ alkyl group substituted with halogen; a C₆-C₁₂ aryl group substituted with halogen; a C₂-C₁₀ heterocyclic group; a C₂-C₁₀ heterocyclic group substituted with halogen; a C₂-C₁₀ heterocyclic group substituted with CF₃; —NR^{a}R^{b}; a —SO₂-phenyl group; a C₆-C₁₂ aryloxy group; a C₂-C₁₂ heteroaryloxy group; and a C₂-C₁₂ heteroaryloxy group substituted with CF₃, and
12) the R^{a} and R^{b} are each independently C₁-C₁₀ alkyl groups.}

2. The compound, the stereoisomer thereof, or the pharmaceutically acceptable salt thereof of claim 1, wherein the R₄ is —SO₂—R₇.

3. The compound, the stereoisomer thereof, or the pharmaceutically acceptable salt thereof of claim 1, wherein the R₃ is a C₁-C₁₀ alkyl group substituted with fluorine.

4. The compound, the stereoisomer thereof, or the pharmaceutically acceptable salt thereof of claim 1, wherein the R₂ is O.

5. The compound, the stereoisomer thereof, or the pharmaceutically acceptable salt thereof of claim 1, wherein the R₁ is NH—R₅.

6. The compound, the stereoisomer thereof, or the pharmaceutically acceptable salt thereof of claim 1, wherein the R₃ is CF₃ or OCF₃.

7. The compound, the stereoisomer thereof, or the pharmaceutically acceptable salt thereof of claim 1, wherein the R₅ is H.

8. The compound, the stereoisomer thereof, or the pharmaceutically acceptable salt thereof of claim 1, wherein the compound is represented by Chemical Formula 2 below. 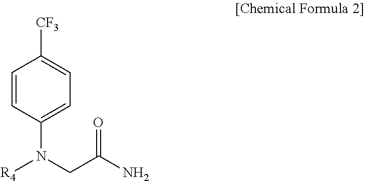
{In Chemical Formula 2, R₄ is the same as the definition of R₄ of claim 1.}

9. The compound, the stereoisomer thereof, or the pharmaceutically acceptable salt thereof of claim 1, wherein the Chemical Formula 1 is any one compound represented by compounds 1-1 to 1-47 below:

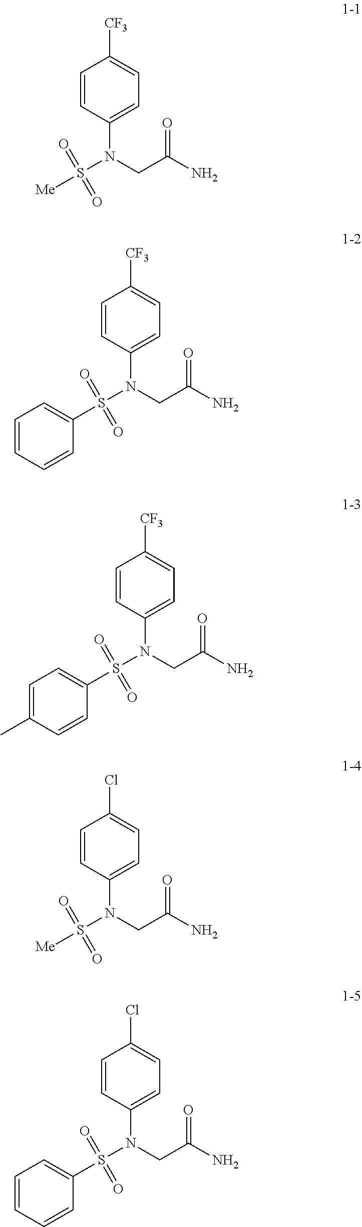 [SMILES: CS(=O)(=O)N(CC(N)=O)c1ccc(C(F)(F)F)cc1 ; NC(=O)CN(c1ccc(C(F)(F)F)cc1)S(=O)(=O)c1ccccc1 ; Cc1ccc(S(=O)(=O)N(CC(N)=O)c2ccc(C(F)(F)F)cc2)cc1 ; CS(=O)(=O)N(CC(N)=O)c1ccc(Cl)cc1 ; NC(=O)CN(c1ccc(Cl)cc1)S(=O)(=O)c1ccccc1]

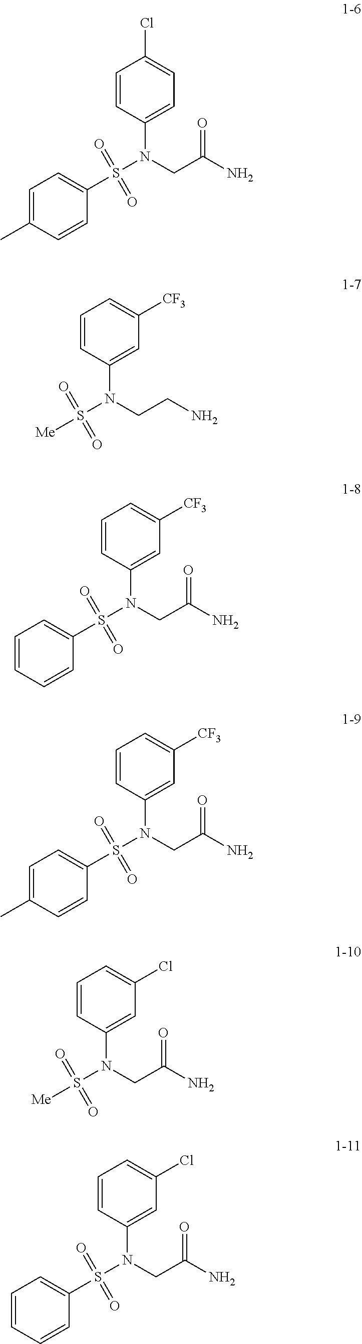 [SMILES: Cc1ccc(S(=O)(=O)N(CC(N)=O)c2ccc(Cl)cc2)cc1 ; CS(=O)(=O)N(CCN)c1cccc(C(F)(F)F)c1 ; NC(=O)CN(c1cccc(C(F)(F)F)c1)S(=O)(=O)c1ccccc1 ; Cc1ccc(S(=O)(=O)N(CC(N)=O)c2cccc(C(F)(F)F)c2)cc1 ; CS(=O)(=O)N(CC(N)=O)c1cccc(Cl)c1 ; NC(=O)CN(c1cccc(Cl)c1)S(=O)(=O)c1ccccc1]

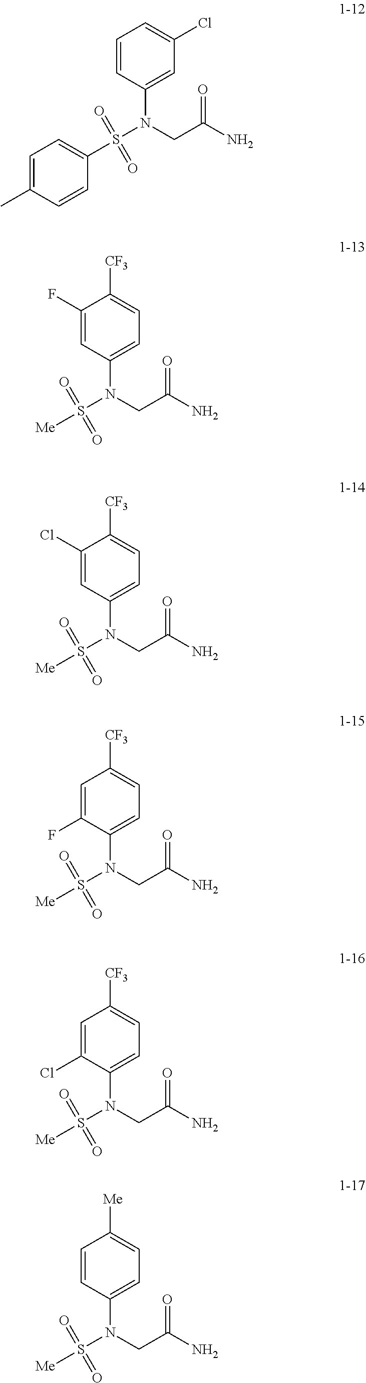 [SMILES: Cc1ccc(S(=O)(=O)N(CC(N)=O)c2cccc(Cl)c2)cc1 ; CS(=O)(=O)N(CC(N)=O)c1ccc(C(F)(F)F)c(F)c1 ; CS(=O)(=O)N(CC(N)=O)c1ccc(C(F)(F)F)c(Cl)c1 ; CS(=O)(=O)N(CC(N)=O)c1ccc(C(F)(F)F)cc1F ; CS(=O)(=O)N(CC(N)=O)c1ccc(C(F)(F)F)cc1Cl ; Cc1ccc(N(CC(N)=O)S(C)(=O)=O)cc1]

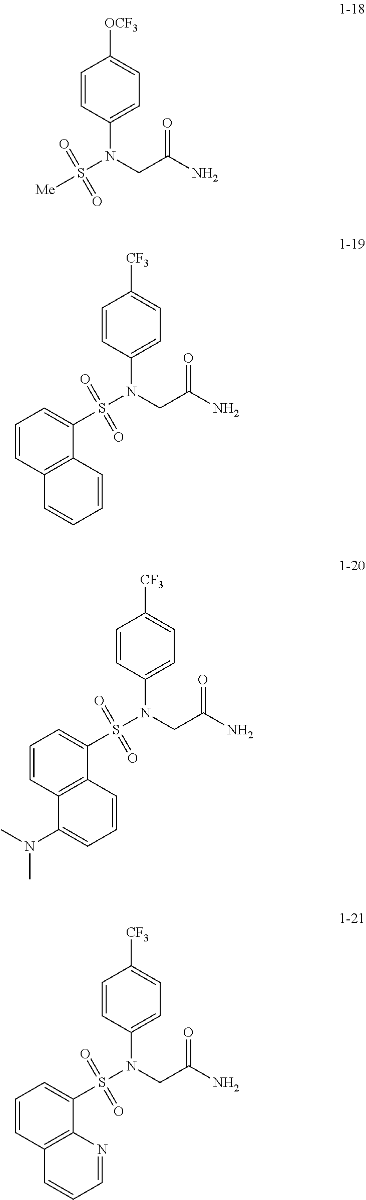 [SMILES: CS(=O)(=O)N(CC(N)=O)c1ccc(OC(F)(F)F)cc1 ; NC(=O)CN(c1ccc(C(F)(F)F)cc1)S(=O)(=O)c1cccc2ccccc12 ; CN(C)c1cccc2c(S(=O)(=O)N(CC(N)=O)c3ccc(C(F)(F)F)cc3)cccc12 ; NC(=O)CN(c1ccc(C(F)(F)F)cc1)S(=O)(=O)c1cccc2cccnc12]

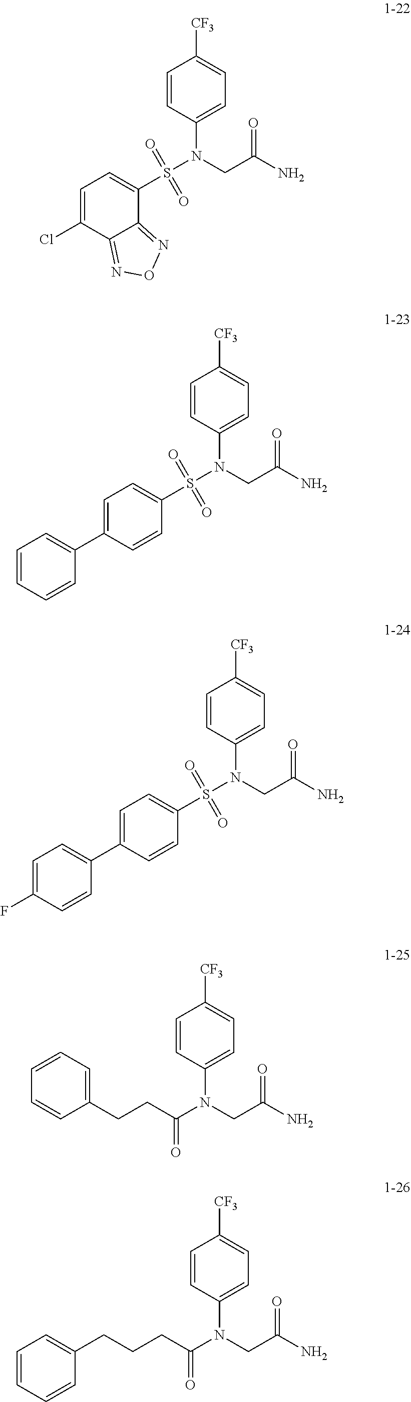 [SMILES: NC(=O)CN(c1ccc(C(F)(F)F)cc1)S(=O)(=O)c1ccc(Cl)c2nonc12 ; NC(=O)CN(c1ccc(C(F)(F)F)cc1)S(=O)(=O)c1ccc(-c2ccccc2)cc1 ; NC(=O)CN(c1ccc(C(F)(F)F)cc1)S(=O)(=O)c1ccc(-c2ccc(F)cc2)cc1 ; NC(=O)CN(C(=O)CCc1ccccc1)c1ccc(C(F)(F)F)cc1 ; NC(=O)CN(C(=O)CCCc1ccccc1)c1ccc(C(F)(F)F)cc1]

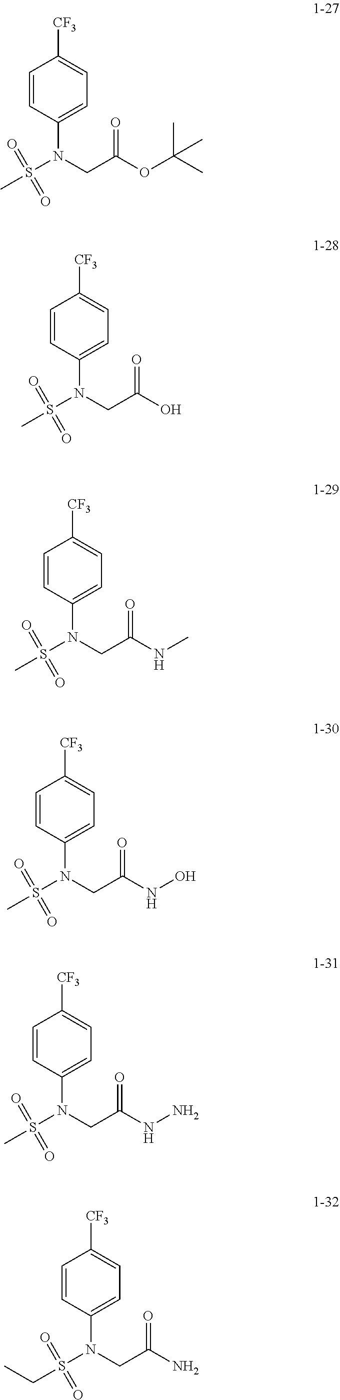

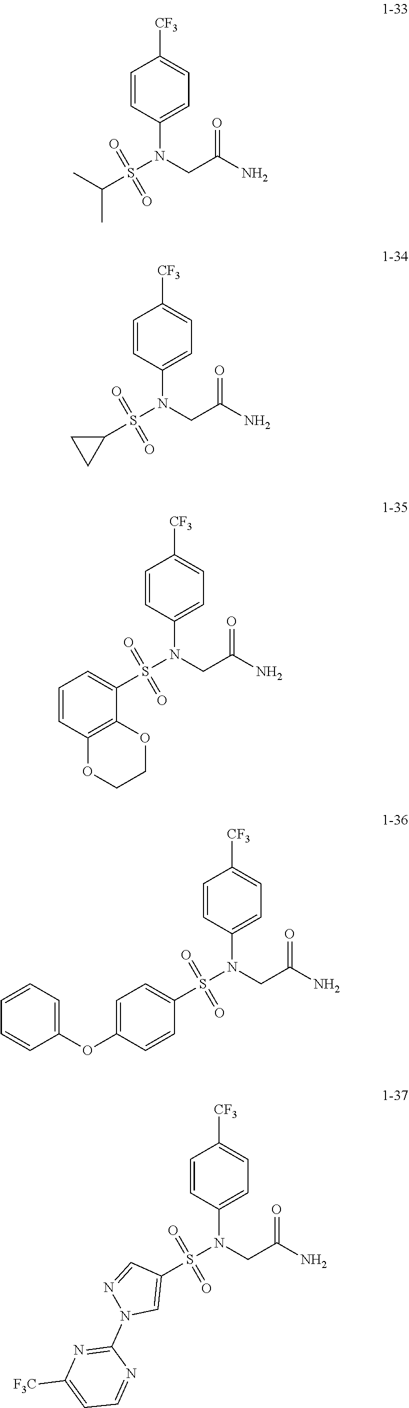 [SMILES: CC(C)S(=O)(=O)N(CC(N)=O)c1ccc(C(F)(F)F)cc1 ; NC(=O)CN(c1ccc(C(F)(F)F)cc1)S(=O)(=O)C1CC1 ; NC(=O)CN(c1ccc(C(F)(F)F)cc1)S(=O)(=O)c1cccc2c1OCCO2 ; NC(=O)CN(c1ccc(C(F)(F)F)cc1)S(=O)(=O)c1ccc(Oc2ccccc2)cc1 ; NC(=O)CN(c1ccc(C(F)(F)F)cc1)S(=O)(=O)c1cnn(-c2nccc(C(F)(F)F)n2)c1]

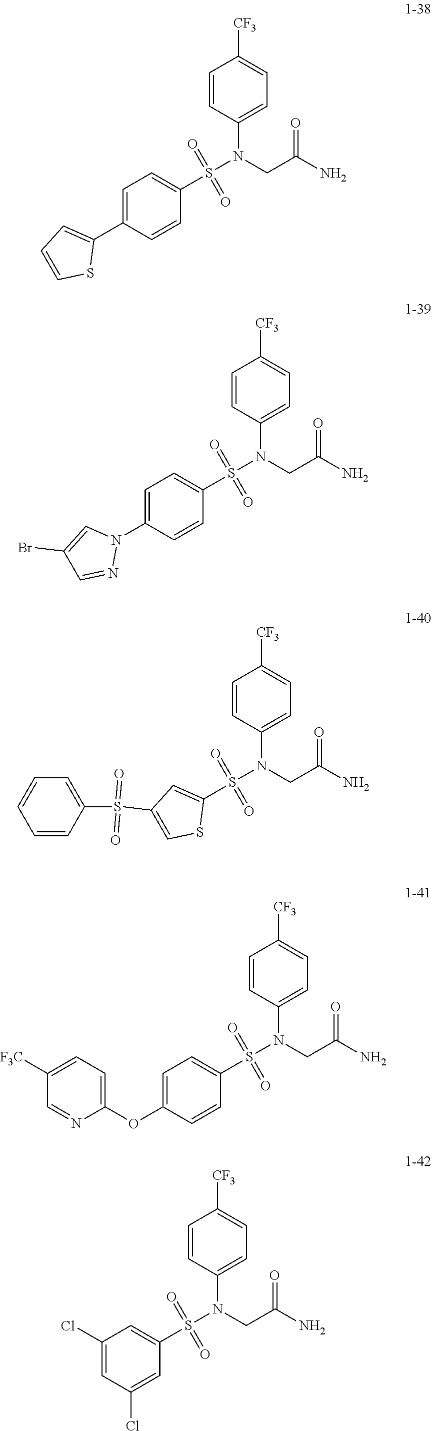 [SMILES: NC(=O)CN(c1ccc(C(F)(F)F)cc1)S(=O)(=O)c1ccc(-c2cccs2)cc1 ; NC(=O)CN(c1ccc(C(F)(F)F)cc1)S(=O)(=O)c1ccc(-n2cc(Br)cn2)cc1 ; NC(=O)CN(c1ccc(C(F)(F)F)cc1)S(=O)(=O)c1cc(S(=O)(=O)c2ccccc2)cs1 ; NC(=O)CN(c1ccc(C(F)(F)F)cc1)S(=O)(=O)c1ccc(Oc2ccc(C(F)(F)F)cn2)cc1 ; NC(=O)CN(c1ccc(C(F)(F)F)cc1)S(=O)(=O)c1cc(Cl)cc(Cl)c1]

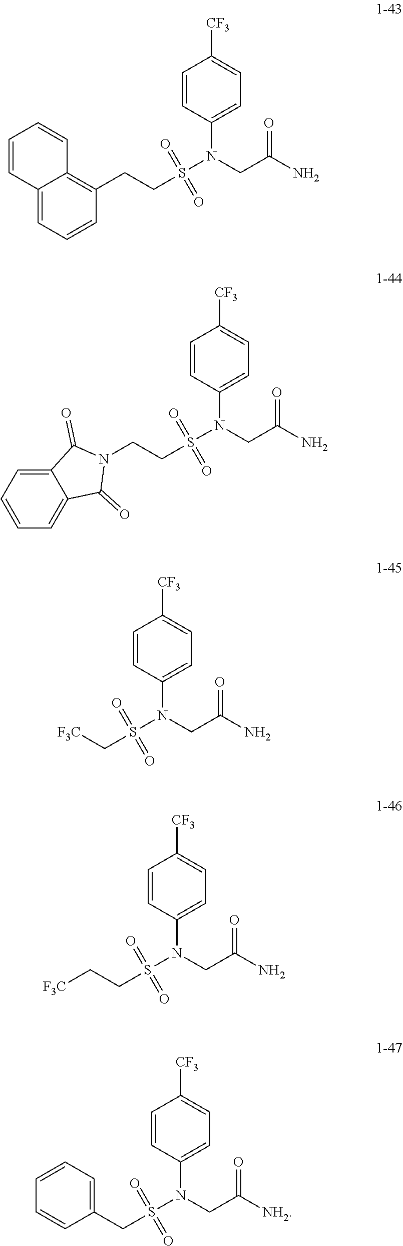 [SMILES: NC(=O)CN(c1ccc(C(F)(F)F)cc1)S(=O)(=O)CCc1cccc2ccccc12 ; NC(=O)CN(c1ccc(C(F)(F)F)cc1)S(=O)(=O)CCN1C(=O)c2ccccc2C1=O ; NC(=O)CN(c1ccc(C(F)(F)F)cc1)S(=O)(=O)CC(F)(F)F ; NC(=O)CN(c1ccc(C(F)(F)F)cc1)S(=O)(=O)CCC(F)(F)F ; NC(=O)CN(c1ccc(C(F)(F)F)cc1)S(=O)(=O)Cc1ccccc1]

10. A pharmaceutical composition comprising the compound represented by Chemical Formula 1 according to claim 1, a stereoisomer thereof, or a pharmaceutically acceptable salt thereof as an active ingredient.

11-17. (canceled)

18. A method for preventing or treating cancer comprising administering the compound represented by Chemical Formula 1 according to claim 1, a stereoisomer thereof, or a pharmaceutically acceptable salt thereof to a subject in need thereof in a therapeutically effective amount.

19-24. (canceled)

25. The pharmaceutical composition of claim 10, further comprising a pharmaceutically acceptable excipient or a pharmaceutically acceptable carrier.

26. The method for preventing or treating cancer of claim 18, wherein the cancer is selected from the group consisting of liver cancer, colorectal cancer, cervical cancer, kidney cancer, gastric cancer, prostate cancer, breast cancer, brain tumor, lung cancer, colon cancer, bladder cancer, and pancreatic cancer.
